(19) United States
(12) Patent Application Publication
(10) Pub. No.: US 2025/0152175 A1
(43) Pub. Date: May 15, 2025

(54) PRESSURE-INDUCED MODULATION SYSTEMS

(71) Applicant: EDWARDS LIFESCIENCES CORPORATION, Irvine, CA (US)

(72) Inventors: Leonardo Paim Nicolau Da Costa, Tampa, FL (US); Emil Karapetian, Huntington Beach, CA (US)

(21) Appl. No.: 19/020,029

(22) Filed: Jan. 14, 2025

Related U.S. Application Data
(63) Continuation of application No. PCT/US2023/071215, filed on Jul. 28, 2023.
(60) Provisional application No. 63/370,632, filed on Aug. 5, 2022.

(51) Int. Cl. A61B 17/12

(52) U.S. Cl. CPC A61B 17/12109; A61B 17/12031; A61B 17/12045; A61B 17/12136; A61B 17/12168

(57) ABSTRACT

A system for modulating blood flow includes a first inflation device sized for placement at least partially within a blood vessel, such as an inferior vena cava, a superior vena cava, or a hepatic vein of a heart. The first inflation device expands and contracts in a cyclic manner. A tube extends from the first inflation device to a second inflation device, which may be placed within a blood vessel or within a chamber of the heart. The tube allows a liquid or gas to pass between the first and second inflation devices. The system may be used to alter the flow rate and/or timing of blood flow. In alternative embodiments, stents may be used in addition to, or in place of, the inflation devices. In some embodiments, a power source may be used with the system to more precisely modulate the blood flow.

### RELATED APPLICATION

This application is a continuation of International Patent Application No. PCT/US2023/071215, filed Jul. 28, 2023, which claims the benefit of U.S. Provisional Application No. 63/370,632, filed Aug. 5, 2022, the disclosures of which are hereby incorporated by reference in their entirety.

### BACKGROUND

The present invention relates generally to the field of medical devices and procedures.

Redistribution of blood from the splanchnic venous circulation to the inferior vena cava (IVC) and/or superior vena cava (SVC) can contribute to increases in central venous pressure (CVP), pulmonary artery pressure, and/or pulmonary capillary wedge pressure (PCWP), particularly during periods of elevated sympathetic tone (e.g., exercise) in heart failure patients.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 provides a schematic representation of portions of the splanchnic circulation.

FIG. 2 provides another schematic representation of the splanchnic circulation, illustrating blood flow from the aorta to the inferior vena cava (IVC).

FIG. 3 illustrates portions of the splanchnic venous circulation acting as a blood reservoir between the aorta and the IVC.

FIG. 4 illustrates an example pressure-induced modulation system comprising a first balloon disposed in the left atrium and a second balloon disposed in the IVC in accordance with one or more examples.

FIG. 5 illustrates a default and/or biased form of an example modulation system, in which a first balloon disposed in the left atrium is in an expanded and/or inflated form and/or a second balloon disposed in the IVC is in a deflated and/or compressed form.

FIG. 6 illustrates an example modulation system in which a first balloon disposed in the left atrium is configured to compress in response to increased left atrial pressure in accordance with one or more examples.

FIG. 7 illustrates another example modulation system implanted in a heart in accordance with one or more embodiments.

FIG. 8 illustrates another modulation system implanted in a heart and comprising one or more balloons in the hepatic veins in accordance with one or more examples.

FIG. 9 illustrates another modulation system implanted in a heart and comprising one or more caval balloons in accordance with one or more examples.

FIGS. 10A and 10B illustrate another modulation system implanted in a heart and comprising one or more balloons in the IVC in accordance with one or more examples.

FIGS. 11A and 11B illustrate an example flow-regulating implant deployed at least partially within an IVC in accordance with one or more embodiments.

FIGS. 12A-12C illustrate another example implant comprising a caval balloon for placement in an IVC and/or other blood vessel in accordance with one or more examples.

FIG. 13 illustrates another example modulation system comprising one or more sensors configured to control one or more means for obstructing, restricting, and/or modulation blood flow into the right atrium in accordance with one or more examples of the present disclosure.

FIG. 14 illustrates an example pressure-induced modulation system comprising a first balloon disposed in the left atrium and a second balloon disposed in the SVC in accordance with one or more examples.

FIG. 15 illustrates an example pressure-induced modulation system comprising a first balloon disposed in the left atrium and a second balloon disposed in the IVC in accordance with one or more examples.

FIGS. 16A and 16B illustrate an example pressure-induced modulation system comprising a first balloon disposed in the left atrium and a second balloon disposed in the SVC in accordance with one or more examples.

FIGS. 17A-17C illustrate an example support device in accordance with one or more examples.

FIGS. 18A and 18B illustrate another example support device in accordance with one or more examples.

FIG. 19 illustrates an example balloon configured for use with one or more support devices (see, e.g., FIG. 20) in accordance with one or more examples.

FIGS. 20A and 20B illustrate an example balloon configured for use with one or more support devices in accordance with one or more examples.

FIGS. 21A and 21B illustrate an example pressure-induced modulation system comprising a first balloon disposed in the left atrium and a second balloon disposed in the SVC in accordance with one or more examples.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

The following includes a general description of human cardiac anatomy that is relevant to certain inventive features and embodiments disclosed herein and is included to provide context for certain aspects of the present disclosure.

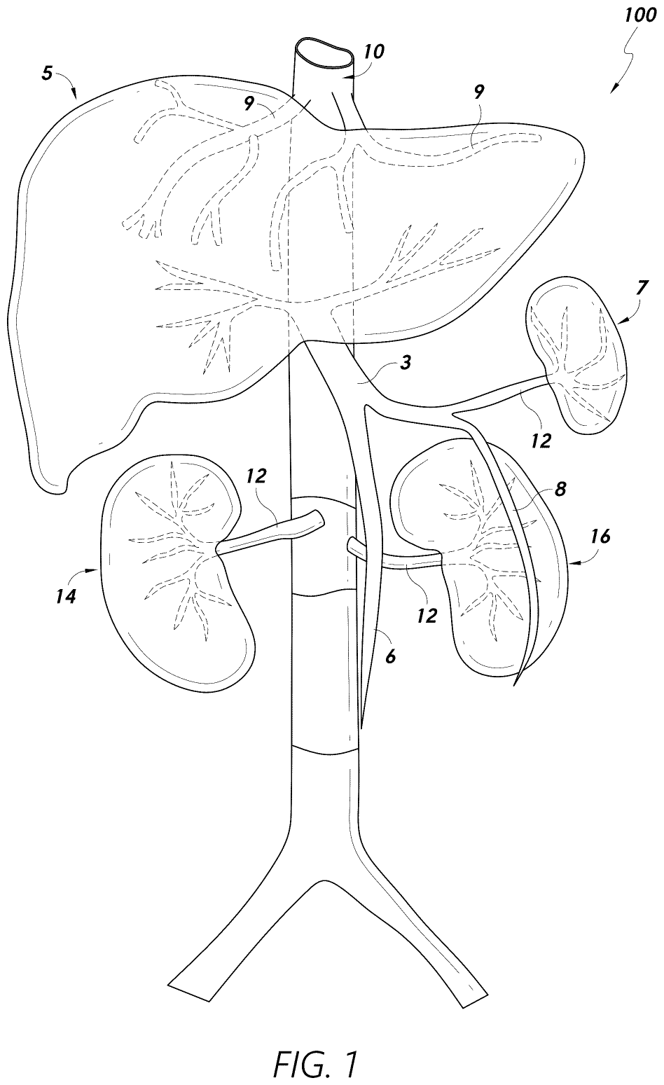

FIG. 1 provides a schematic representation of portions of the splanchnic circulation 100. The term “splanchnic circulation” refers to blood flow originating from the celiac, superior mesenteric, and inferior mesenteric arteries to the abdominal gastrointestinal organs. The splanchnic circulation 100 receives approximately 25% of the cardiac output and holds a similar percentage of the total blood volume under normal conditions. The splanchnic circulation 100 can act as a site of cardiac output regulation and/or as a blood reservoir. Multiple regulatory pathways are involved in the distribution of the splanchnic circulation.

Total flow to the splanchnic viscera is controlled by resistance vessels in the mesenteric and hepatic arterial systems. The venous effluents from the splanchnic viscera converge to form the portal vein 3, which supplies approximately 75% of the total blood supply to the liver 5. The portal blood not only is high in substrate concentrations resulting from intestinal absorption but also tends to contain bacteria and endotoxin.

Renal veins 12 drain blood from the right kidney 14 and left kidney 16 and connect to the inferior vena cava 10 (IVC). The superior mesenteric vein 6 is a major venous tributary of the abdominal cavity that lies laterally to the superior mesenteric artery and serves to drain the vast majority of the organs of the abdominal cavity. The inferior mesenteric vein 8 drains blood from the large intestine. The splenic vein 12 is a blood vessel that drains blood from the spleen, the stomach fundus, and part of the pancreas.

The portal vein 3 receives blood from the stomach, intestines, pancreas, and spleen 7 and carries it into the liver 5 through the porta hepatis. The porta hepatis serves as the point of entry for the portal vein 3 and the proper hepatic artery, and is the point of exit for the bile passages.

Following processing of the blood by the liver 5, the blood collects in the central vein at the core of the lobule. Blood from these central veins ultimately converges in the right and left hepatic veins 9, which exit the superior surface of the liver 5 and empty into the IVC 10 to be distributed to the rest of the body.

The splanchnic venous circulation 100 is highly compliant and can act as a blood reservoir that can be recruited in order to support the need for increased stressed blood volume during periods of elevated sympathetic tone, such as exertion, in order to support increased cardiac output and vasodilation of peripheral vessels supporting active muscles. However, heart failure patients can have multiple comorbidities that prevent them from using that additional blood volume. Such comorbidities can include chronotropic incompetence, inability to increase stroke volume, and/or peripheral microvascular dysfunction. This can lead to venous congestion and/or abrupt rises in pulmonary capillary wedge pressure (PCWP).

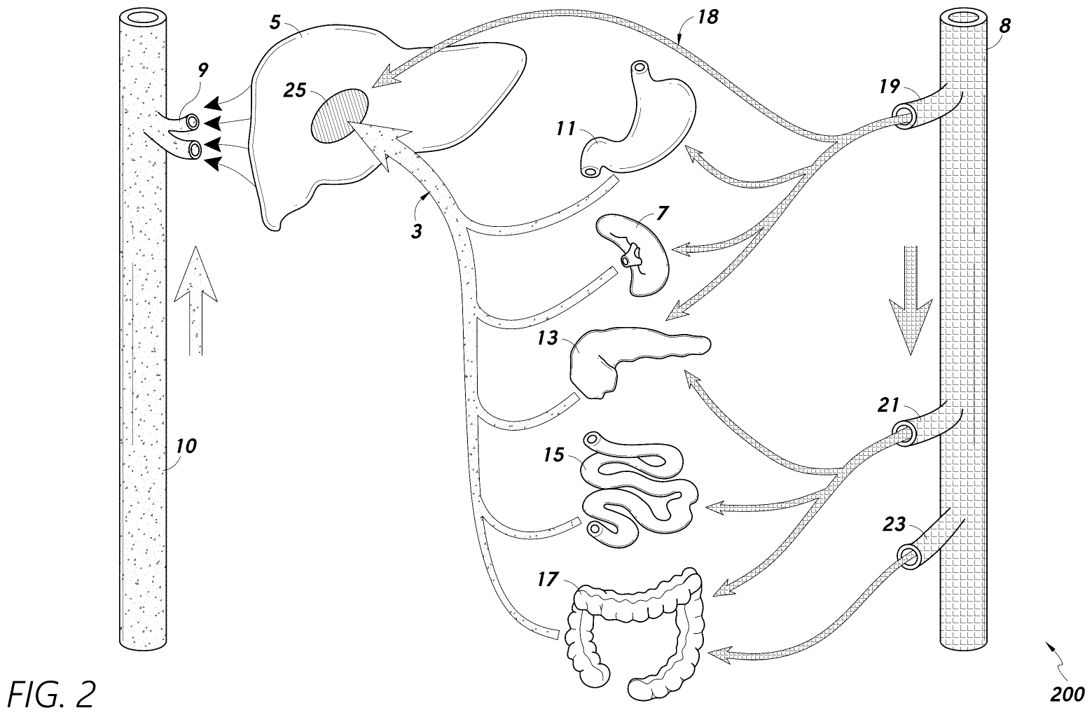

FIG. 2 provides another schematic representation of the splanchnic circulation 200, illustrating blood flow from the aorta 8 to the IVC 10. Blood travels from the aorta 8 to the abdominal gastrointestinal organs including the stomach 11, liver 5, spleen, 7, pancreas 13, small intestine 15, and large intestine 17. The splanchnic circulation 200 comprises three major branches of the abdominal aorta 9, including the coeliac artery 19, the superior mesenteric artery 21 (SMA), and the inferior mesenteric artery 23 (IMA). The hepatic portal circulation (e.g., the hepatic artery 18 and/or portal vein 3) delivers the majority of blood flow to the liver 5.

The coeliac artery 19 is the first major division of the abdominal aorta 8, branching at T12 in a horizontal direction ˜1.25 cm in length. It shows three main divisions such as the left gastric artery, common hepatic artery 18, and splenic artery and is the primary blood supply to the stomach 11, upper duodenum, spleen 7, and pancreas 13.

The SMA 21 arises from the abdominal aorta 8 anteriorly at L1, usually 1 cm inferior to the coeliac artery 19. The five major divisions of the SMA 21 are the inferior pancreaticoduodenal artery, intestinal arteries, ileocolic, right colic, and middle colic arteries. The SMA 21 supplies the lower part of the duodenum, jejunum, ileum, caecum, appendix, ascending colon, and two-thirds of the transverse colon. It is the largest of the splanchnic arterial vessels delivering >10% of the cardiac output and therefore has significant implications for embolic mesenteric ischaemia.

The IMA 23 branches anteriorly from the abdominal aorta 8 at L3, midway between the renal arteries and the iliac bifurcation. The main branches of the IMA 23 are the left colic artery, the sigmoid branches, and the superior rectal artery. It forms a watershed with the middle colic artery and supplies blood to the final third of the transverse colon, descending colon, and upper rectum.

Blood flow is conveyed into the liver 5 via the portal vein 3 into sinusoids 25 of the liver 5. The hepatic veins 9 convey the blood from the liver 5 to the IVC 10.

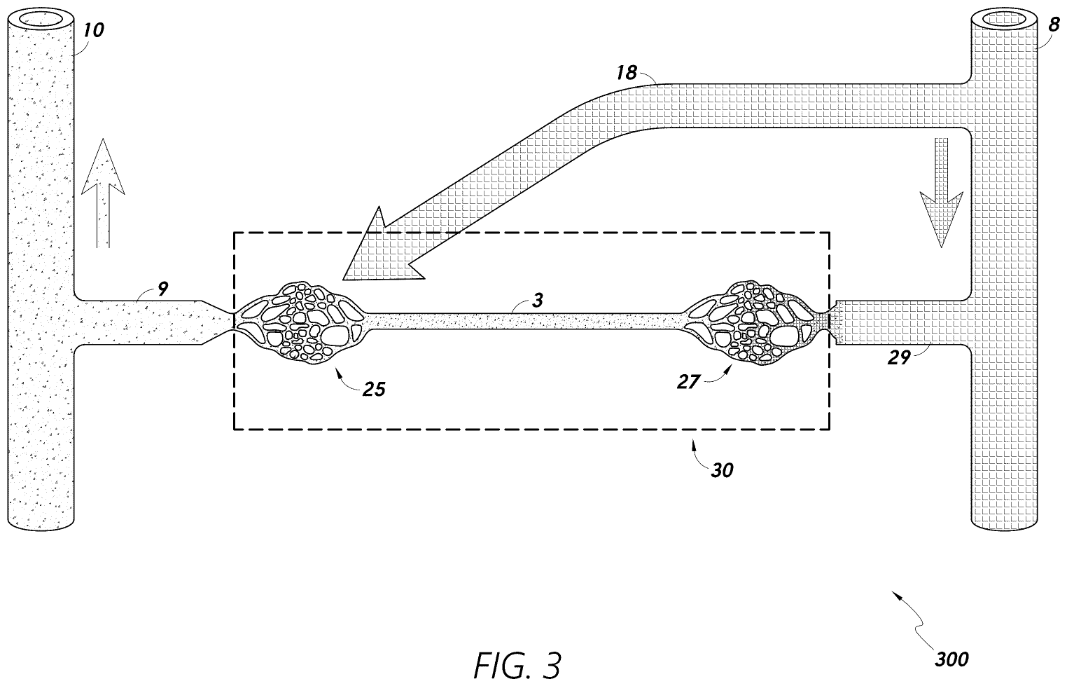

FIG. 3 illustrates portions of the splanchnic venous circulation 300 acting as a blood reservoir 30 between the aorta 8 and the IVC 10. The portal vein 30 conveys blood between the splanchnic organs 27 (e.g., the stomach, spleen, etc.) and the liver sinusoids 25. The liver sinusoids 25 also receive blood from the hepatic artery 18. The splanchnic organs 27 receive blood from the aorta 8 via various splanchnic arteries 29 (e.g., the SMA, IMA, etc.). The amount of blood contained in the portal vein 3 at any given time can be variable.

For some patients (especially patients experiencing heart failure) fluid redistribution from the splanchnic venous reservoir 30 to the IVC 10 and/or stressed blood volume can contribute to increases in central venous pressure (CVP), pulmonary artery pressure, and/or PCWP. This can be especially problematic during periods of elevated sympathetic tone, such as exertion, and/or can lead to pulmonary congestion that can impact a patient's quality of life and/or can lead to acute decompensation.

The splanchnic venous circulation 300, and particularly the portal vein 3, can advantageously provide a blood reserve to support the need for increased stressed blood volume during periods of elevated sympathetic tone. Because blood flow from the splanchnic venous circulation 300 is directed through the hepatic veins 9 and into the IVC 10, devices placed into the hepatic veins 9 and/or IVC 10 to limit blood flow can allow the reservoir 30 to expand with increased blood volume.

Embodiments described herein can relate to devices and/or methods that can advantageously limit, stagnate, and/or impede blood flow into the IVC 10 from the hepatic veins 9 to increase the pressure gradient between the IVC 10 and the liver and/or splanchnic venous circulation 300. In some embodiments, one or more flow-regulating implants may be configured for placement at least partially within the hepatic veins 9 and/or IVC 10 and/or at one or more junctions between the hepatic veins 9 and the IVC 10. As a result, blood flowing from the splanchnic venous reservoir 30 into the hepatic veins 9 can be slowed to increase blood volume in the splanchnic venous reservoir 30.

Some approaches to reducing volume redistribution can involve placing fixed orifice flow restrictors at or near the IVC 10. However, while restricting the flow from the hepatic veins 9 can be beneficial in preventing volume redistribution, too much restriction can cause hepatic congestion. It would therefore be advantageous to modulate the response and increase restriction only during volume redistribution.

Some examples presented herein relate to methods and/or devices for increasing the restriction of blood flow from the hepatic veins 9 and/or IVC 10 into the right atrium as pressures increase in the left atrium. In some instances, a device can comprise two or more interconnected balloons and/or similar devices. The term “balloon” is used herein in accordance with its plain and ordinary meaning and may refer to any inflatable, deflatable, compressible, expandable, and/or fillable device. A balloon may be configured to be inflated and/or filled with a gas, liquid, and/or similar substance.

In some examples, a first balloon may be disposed at least partially within the left atrium and/or a second balloon may be disposed at least partially in the IVC 10. One or more balloons within the IVC 10 may be disposed at or near a level of one or more hepatic veins 9. One or more balloons can be filled with fluid and/or gas and/or can modulate preload pressures in the heart by alternately expanding and/or compressing. In some examples, one balloon may be inflated while another balloon is deflated.

The various devices can be implanted using a transcatheter transvenous approach, for example entering through the femoral vein. With a transeptal puncture, the left atrium may be accessed, and one or more balloons can be deployed in the left atrium. Alternatively, one or more balloons may be deployed in the left atrium via navigation through the coronary sinus. The delivery system can then be progressively unsheathed, and other components of the device can be sequentially implanted under fluoroscopic and echo guidance, if necessary.

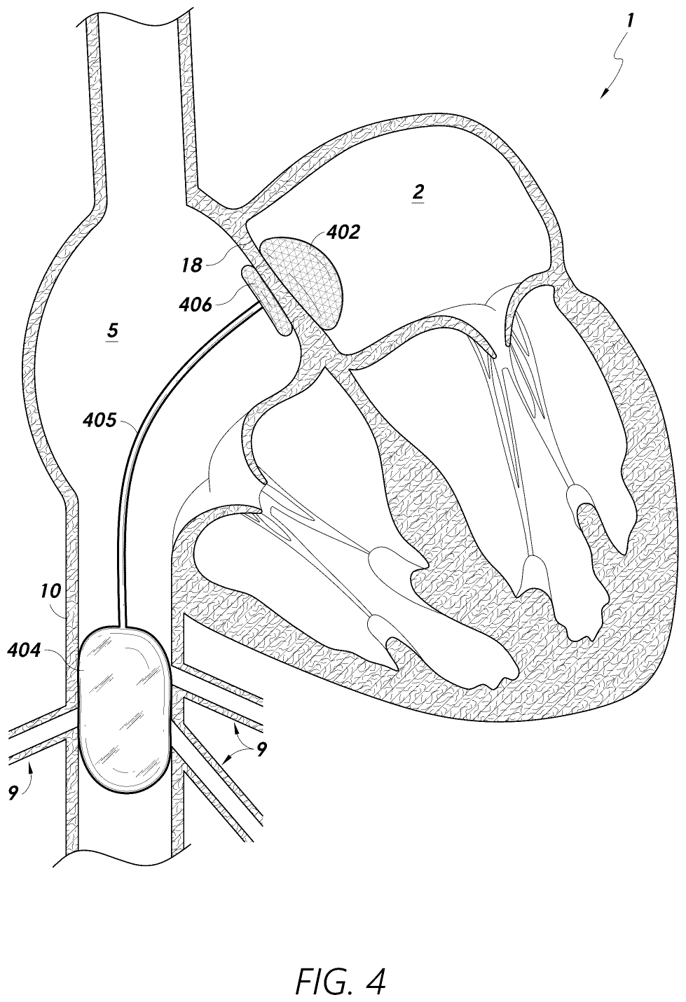

FIG. 4 illustrates an example pressure-induced modulation system comprising a first balloon 402 disposed in the left atrium 2 and a second balloon 404 disposed in the IVC 10 in accordance with one or more examples. The first balloon 402 and the second balloon 404 may be interconnected via a tube 405, which may include any suitable device configured for conveying liquid and/or gas between the first balloon 402 and the second balloon 404. In some examples, the tube 405 may have a generally thin and/or elongate tubular form. The tube 405 may provide a fluidic connection and/or channel between the first balloon 402 and the second balloon 404. For example, as a fluid and/or gas is pressed out of the first balloon 402, the fluid and/or gas may be passed from the first balloon 402 to the second balloon 404, and vice versa.

In some examples, the tube 405 may connect to and/or extend from the first balloon 402 and the second balloon 404 via an open channel and/or a selectively closed channel. For example, one or more flaps may be used to selectively allow flow from the first balloon 402 and/or the second balloon 404 into the tube 405 and/or vice versa. In some examples, the tube 405 may be configured to be disposed at least partially within the right atrium 5 and/or IVC 10.

In some examples, the first balloon 402 can have a dome and/or partial spherical (e.g., hemispherical) shape. For example, the first balloon 402 may comprise half of a sphere and/or may have a generally flat base portion configured to rest against a surface of the atrial septum 18. As the first balloon 402 extends away from the septum 18, the shape of the first balloon 402 may become more rounded. However, the first balloon 402 may have any suitable shape. For example, the first balloon 402 may alternatively comprise a generally circular and/or ovular shape.

The first balloon 402 may be secured to the septum 18 in any suitable manner. In some examples, a support anchor 406 may be configured to anchor the first balloon 402 in place. The anchor 406 may comprise a generally solid and/or hollow structure configured to be disposed opposite the first balloon 402 in the right atrium 5 and/or against the septum 18 on the right atrium 5 side. At least a portion of the septum 18 may be sandwiched between the anchor 406 and the first balloon 402. In some examples, the anchor 406 may comprise an inflatable balloon and/or other expandable device. The anchor 406 may be connected to the first balloon 402 and/or may be an extension of the first balloon 402. Alternatively, the anchor 406 may be a separate device and/or may be joined to the first balloon 402 via the tube 405 and/or other tethering device. In some examples, the anchor 406 may comprise a disc and/or similar device and/or may have a generally circular and/or ring-shaped form.

In some examples, the tube 405 may be configured to extend at least partially through the anchor 406 and/or through the septum 18 as the tube 405 extends from the first balloon 402 towards the IVC 10. The anchor 406 and/or first balloon 402 may comprise apertures and/or openings configured to receive and/or accommodate the tube 405.

The second balloon 404 may be delivered into the heart 1 separately and/or together with the first balloon 402. In some examples, the second balloon 404 may have a generally spherical form and/or may be configured to approximate a cross-sectional shape of the IVC 10. The tube 405 may have a suitable length such that the second balloon 404 may be configured to extend at least partially into the IVC 10 and/or in front of inflow channels from one or more hepatic veins 9. In some examples, the second balloon 404 may be configured to inflate to a diameter that is greater than the one or more hepatic veins 9 and/or approximately equal to or greater than a diameter of the IVC 10.

The first balloon 402, second balloon 404, and/or anchor 406 may have any suitable structure. In some examples, the first balloon 402, second balloon 404, and/or anchor 406 may comprise an outer layer and/or an inner layer. The inner layer may comprise a generally flexible, stretchy, and/or elastic material, which can include rubber, latex, and/or similar materials. The outer layer may have a generally rigid and/or bendable structure and/or may comprise one or more interwoven wires and/or lines of material. The outer layer may be at least partially composed of Nitinol and/or other shape memory alloys.

The first balloon 402 and/or second balloon 404 may be configured to be filled with an incompressible fluid. In some examples, the first balloon 402 and/or second balloon 404 may be implanted through an endovascular transeptal approach.

In some examples, the first balloon 402 and/or the second balloon 404 may comprise various features configured to regulate inflation and/or deflation and/or to regulate a rate of inflation and/or deflation of the first balloon 402 and/or the second balloon 404. For example, one or more springs, coils, and/or similar elements (e.g., Nitinol wire forms) may be attached to an exterior of the first balloon 402 and/or second balloon 404 and/or may be placed inside the first balloon 402 and/or the second balloon 404 to regulate the rate of deflation and/or inflation.

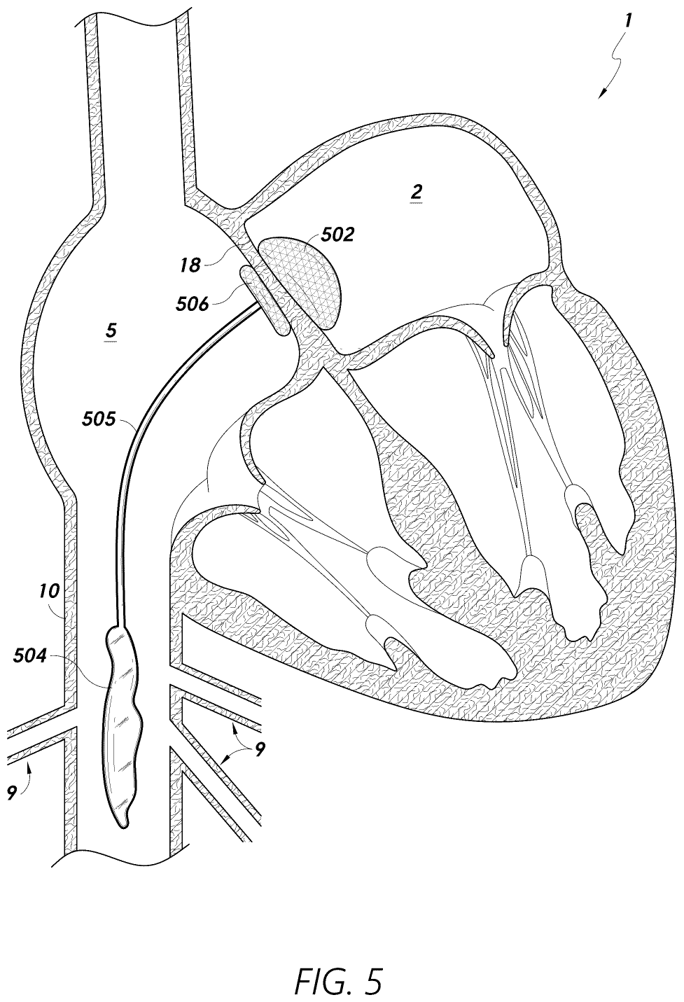

FIG. 5 illustrates a default and/or biased form of an example modulation system, in which a first balloon 502 disposed in the left atrium 2 is in an expanded and/or inflated form and/or a second balloon 504 disposed in the IVC 10 is in a deflated and/or compressed form. The first balloon 502 may be biased towards an expanded and/or inflated form. For example, when there is relatively low and/or normal blood pressure within the left atrium 2, the first balloon 502 may be configured to maintain the expanded and/or inflated form. The second balloon 504 may be biased toward a deflated and/or unexpanded state. For example, when there is relatively low and/or normal blood pressure within the left atrium 2 and/or IVC 10, the second balloon 504 may be configured to maintain a deflated and/or compressed form.

The first balloon 502 and the second balloon 504 may be fluidically connected via one or more tubes 505 configured to convey gas and/or fluid into and/or between the first balloon 502 and/or the second balloon 504. While only a single tube 505 is shown in FIG. 5, multiple tubes 505 may be used to facilitate exchange of gas and/or fluid between the first balloon 502 and the second balloon 504.

In some examples, the system may comprise one or more anchors 506 configured to anchor the first balloon 502 and/or tube 505 in place and/or in a desired position. For example, an anchor 506 may be configured to hold the first balloon 502 against the septum 18 and/or within the left atrium 2. In some examples, an anchor 506 may be disposed in the right atrium 5 and/or in contact with the right atrium 5 side of the septum 18. The anchor 506 may have any suitable size and/or shape. For example, the anchor 506 may have a form of a disc.

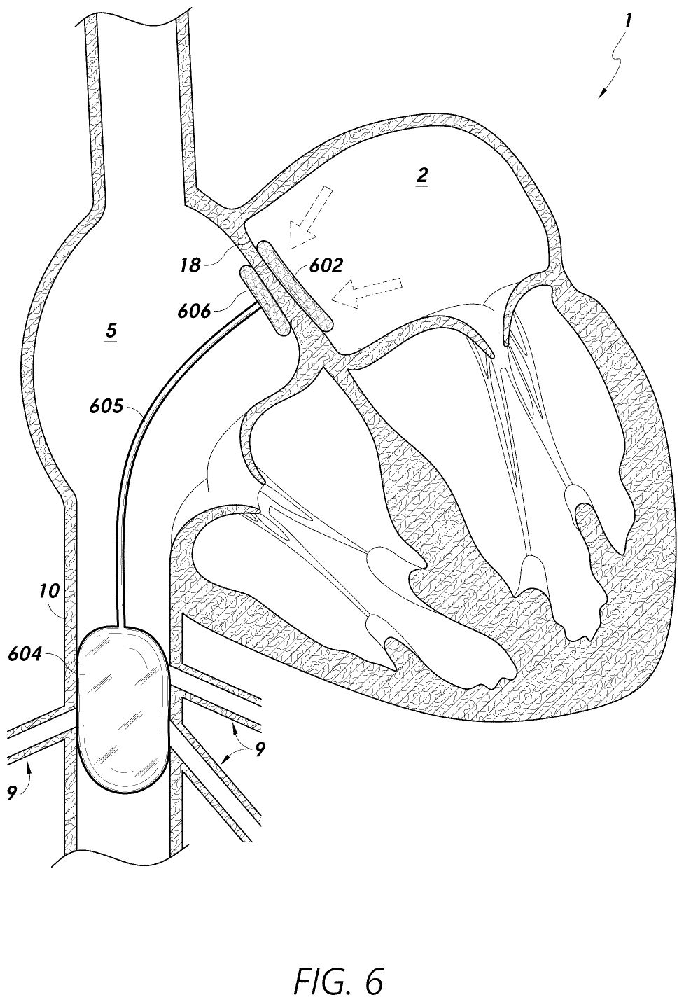

FIG. 6 illustrates an example modulation system in which a first balloon 602 disposed in the left atrium 2 is configured to compress in response to increased left atrial pressure in accordance with one or more examples. As an extra preload volume of blood enters the circulatory system through the IVC 10 and hepatic veins 9, pressure in the left atrium 2 increases. As a result of increased left atrium pressure, the first balloon 602 may be configured to at least partially compress and/or deflate, as shown in FIG. 6. Compression of the first balloon 602 may cause shifting of a volume of gas and/or liquid (e.g., saline) out of the first balloon 602 and/or into a second balloon 604 situated in the IVC 10. The second balloon 604 may be configured to inflate and/or expand in response to the influx of gas and/or fluid from the first balloon 602. Inflation of the second balloon 604 can create increased blockage and/or obstruction of the IVC 10 and/or hepatic veins 9, which can reduce the inflow of blood from the IVC 10 and/or hepatic veins 9 into the right atrium 6. As a result cardiac preload and/or chamber filling pressures can be modulated.

As the left atrial pressure returns to normal, the first balloon 602 and/or second balloon 604 can be configured to return to the forms shown in FIG. 5 due to the pre-existing biases in the first balloon 602 and/or second balloon 604. As a result, blood flow through the IVC 10 may be unrestricted in a resting state and/or may maintain normal physiology.

The first balloon 602, second balloon 604 and/or tube 605 may be configured to be internally filled with a bio-inert incompressible fluid, such as saline. Deflation of one balloon can be configured to cause subsequent inflation of the other balloon. For example, during use, the system can alternate between the resting state of FIG. 5 and an active state illustrated in FIG. 6, in which the first balloon is deflated and the second balloon is inflated.

In some examples, one or more anchors 606 may be configured to anchor the first balloon 602 and/or the tube 605 in place and/or at desired positions. The one or more anchors 606 may be generally hollow and/or may be inflated with gas and/or fluid conveyed via the tube 605. Alternatively, the one or more anchors 606 may be generally solid and/or may be configured to remain constantly inflated. For example, when the first balloon 602 is deflated and/or compressed, the one or more anchors 606 may remain in a constant expanded form.

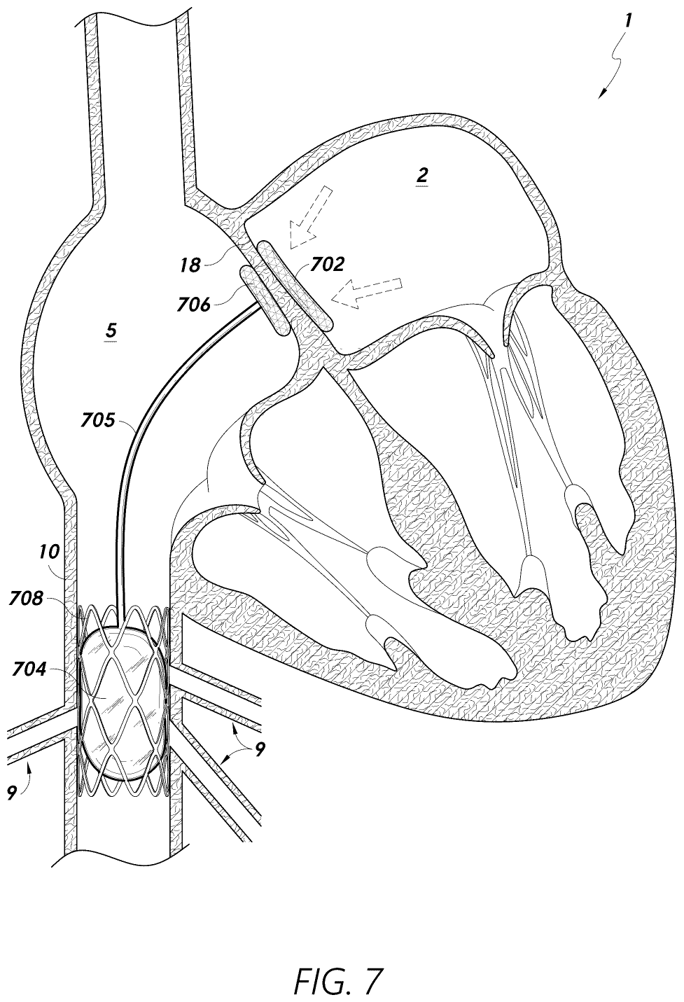

FIG. 7 illustrates another example modulation system implanted in a heart 1 in accordance with one or more embodiments. The modulation system may be pressure-induced and/or may comprise a first balloon 702 disposed in the left atrium 2 and a second balloon 704 disposed in the IVC 10 in accordance with one or more examples. The first balloon 702 and the second balloon 704 may be interconnected via a tube 705, which may include any suitable device configured for conveying liquid and/or gas between the first balloon 702 and the second balloon 704. In some examples, the tube 705 may have a generally thin and/or elongate tubular form. The tube 705 may provide a fluidic connection and/or channel between the first balloon 702 and the second balloon 704. For example, as a fluid and/or gas is pressed out of the first balloon 702, the fluid and/or gas may be passed from the first balloon 702 to the second balloon 704, and vice versa.

In some examples, the second balloon 704 may be held in place and/or at least partially enclosed by a stent 708 and/or cage-like structure. The stent 708 may be configured to prevent the second balloon 704 from migrating up into the right atrium 5 and/or further down into the IVC 10. In some examples, the stent 708 may comprise an uncovered wire frame forming struts that may allow some blood flow through the wire frame.

The stent 708 may have a generally cylindrical and/or tubular form and/or may be configured to approximate a shape and/or size of the IVC 10. In some examples, the stent 708 may have a length that may be greater than the second balloon 704 such that the stent 708 may be configured to fully enclose the second balloon 704 lengthwise as well as circumferentially.

In some examples, the stent 708 may be configured to extend at least partially across and/or in front of one or more hepatic veins 9 branching into the IVC 10. The stent 708 may be at least partially porous and/or may be configured to form cells through which blood flow from the hepatic veins 9 can flow through. The stent 708 may be configured to have no or minimal effect on blood flow from the hepatic veins 9. As the second balloon 704 inflates and/or expands, the second balloon 704 may be configured to close cells formed by the stent 708 to potentially obstruct and/or limit blood flow from the hepatic veins 9.

The stent 708 may be expandable (e.g., balloon expandable) and/or may be configured to be expanded following delivery into the IVC 10. In some examples, a separate expansion balloon may be used to expand the stent 708. However, expansion and/or inflation of the second balloon 704 may be configured to cause expansion of the stent 708. For example, the second balloon 704 may be placed into the stent 708 in a compressed and/or uninflated state. When the second balloon 704 expands, the second balloon 704 may cause expansion of at least a portion of the stent 708. In some examples, the stent 708 may have a generally non-elastic structure and/or may be configured to maintain an expanded form following expansion by the second balloon 704.

In some examples, the stent 708 may be configured to attach to the second balloon 704 and/or to the tube 705. For example, the second balloon 704 and/or stent 708 may comprise one or more tethers, hooks, latches, anchors, and/or other features configured to establish one or more connections between the second balloon 704 and the stent 708. As a result, the second balloon 704 may be prevented from migrating relative to the stent 708.

The stent 708 may comprise one or more anchoring features (e.g., anchors) configured to anchor the stent 708 to the IVC 10. For example, the stent 708 may comprise one or more hooks, arms, and/or similar features configured to anchor into tissue of the IVC 10. However, the stent 708 may be configured to anchor to the IVC 10 independently of anchors. For example, the stent 708 may be configured to expand to a diameter that is approximately equal to and/or greater than the diameter of the IVC 10 such that the stent 708 may be configured to expand to a size that presses against the walls of the IVC 10 and holds the stent 708 in place.

The system may comprise one or more anchors 706 configured to anchor the first balloon 702 in place. The one or more anchors 706 may have a shape and/or size configured to prevent the one or more anchors 706 from passing through an opening in the septum 18.

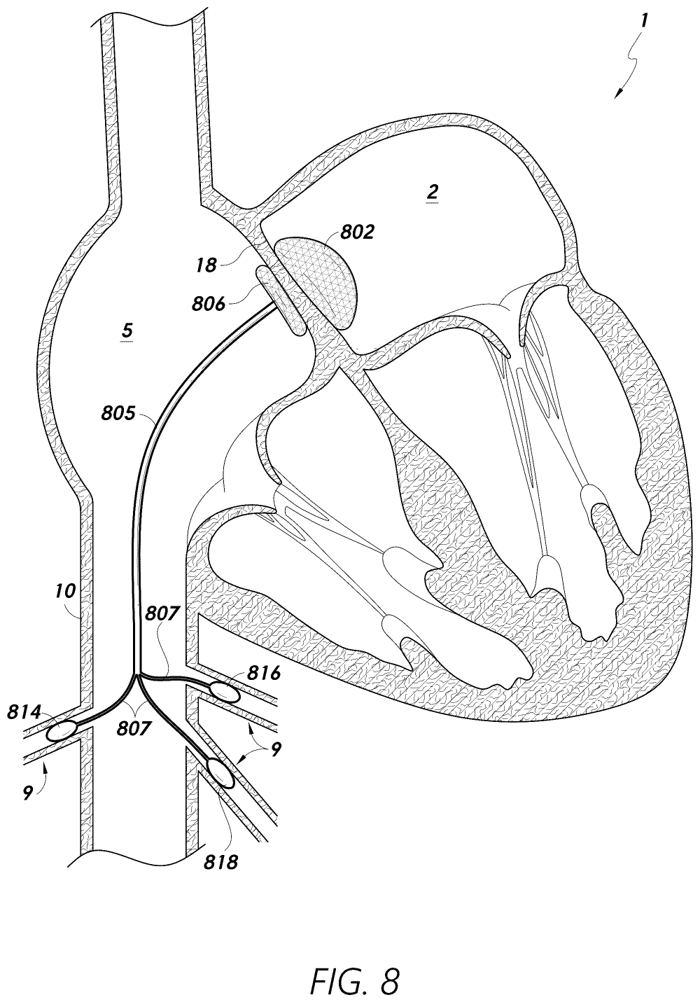

FIG. 8 illustrates another modulation system implanted in a heart 1 and comprising one or more balloons in the hepatic veins in accordance with one or more examples. The modulation system may be pressure-induced and/or may comprise a first balloon 802 disposed in the left atrium 2 and one or more balloons, which can include a second balloon 814, a third balloon 816, and/or a fourth balloon 818 disposed in the IVC 10 and/or hepatic veins 9 in accordance with one or more examples. The first balloon 802, the second balloon 814, the third balloon 816, and/or the fourth balloon 818 may be interconnected via a tube 805, which may include any suitable device configured for conveying liquid and/or gas between the balloons. In some examples, the tube 805 may have a generally thin and/or elongate tubular form. The tube 805 may provide a fluidic connection and/or channel between the balloons. For example, as a fluid and/or gas is pressed out of the first balloon 802 (e.g., in response to increased pressure in the left atrium 2, the fluid and/or gas may be passed from the first balloon 802 to the second balloon 814, third balloon 816, and/or fourth balloon 818. Similarly, as pressure in the left atrium 2 drops, fluid and/or gas form the second balloon 814, third balloon 816, and/or fourth balloon 818 may be pressed into the tube 805 and/or back into the first balloon 802.

In some examples, the second balloon 814, third balloon 816, and/or fourth balloon 818 may be disposed at least partially within hepatic veins 9 and/or may not be disposed within and/or extend into the IVC 10. In this way, the second balloon 814, third balloon 816, and/or fourth balloon 818 may advantageously avoid obstructing the IVC 10 while limiting inflow from the hepatic veins 9. The second balloon 814, third balloon 816, and/or fourth balloon 818 may have generally smaller diameters than balloons described herein for placement within the IVC 10. For example, a diameter of a hepatic vein 9 may be smaller than a diameter of the IVC 10. Accordingly, the second balloon 814, third balloon 816, and/or fourth balloon 818 may be configured to expand to smaller diameters than expanded forms of other balloons described herein.

While the second balloon 814, third balloon 816, and/or fourth balloon 818 are shown without enclosures (e.g., stents), the second balloon 814, third balloon 816, and/or fourth balloon 818 may be at least partially enclosed by one or more stents and/or may otherwise be anchored within the hepatic veins 9 to prevent the second balloon 814, third balloon 816, and/or fourth balloon 818 from migrating out of the hepatic veins 9 and/or deeper into the hepatic veins 9. The second balloon 814, third balloon 816, and/or fourth balloon 818 may be configured to be positioned at or near branching ends of the hepatic veins 9 and/or near the IVC 10. In some examples, the system may comprise a stent placed in the IVC 10 to prevent the second balloon 814, third balloon 816, and/or fourth balloon 818 from exiting the hepatic veins 9 and/or entering the IVC 10. For example, a cylindrical stent may be placed in the IVC 10 and/or may extend across inflow junctions of one or more hepatic veins 9. The stent may be delivered before and/or following placement of the second balloon 814, third balloon 816, and/or fourth balloon 818 in the hepatic veins 9.

In some examples, the system may comprise multiple tubes 805 and/or the tube 805 may be configured to branch into multiple branching tubes 807 which may individually extend to the various hepatic vein 9 balloons. Branching tubes 807 may have smaller diameters than the main tube 805.

The system may comprise one or more anchors 806 configured to anchor the first balloon 802 in place. The one or more anchors 806 may have a shape and/or size configured to prevent the one or more anchors 806 from passing through an opening in the septum 18.

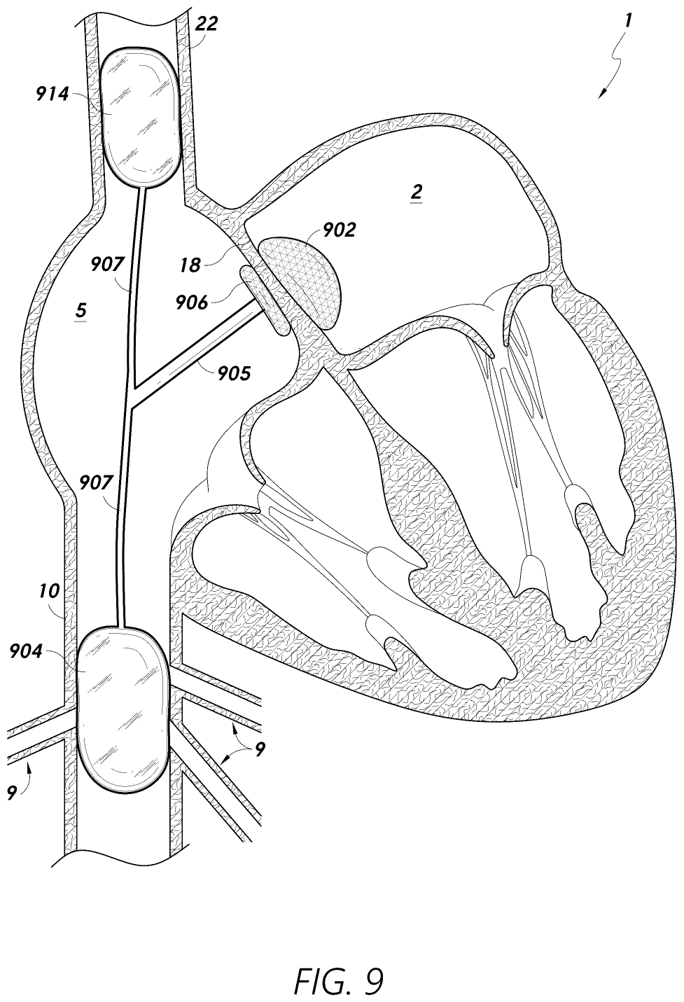

FIG. 9 illustrates another modulation system implanted in a heart 1 and comprising one or more caval balloons in accordance with one or more examples. The modulation system may be pressure-induced and/or may comprise a first balloon 902 disposed in the left atrium 2 and one or more balloons, which can include a second balloon 904 and/or a third balloon 914, configured to obstruct blood flow into the right atrium 5. The first balloon 902, the second balloon 904, and/or the third balloon 914 may be interconnected via a tube 905 and/or via one or more branching tubes 907 extending from a main tube 905. The tube 905 and/or branching tubes 907 may include any suitable device configured for conveying liquid and/or gas between the balloons. In some examples, the tube 905 and/or branching tubes 907 may have generally thin and/or elongate tubular forms. The tube 905 and/or branching tubes 907 may provide a fluidic connection and/or channel between the balloons. For example, as a fluid and/or gas is pressed out of the first balloon 902 (e.g., in response to increased pressure in the left atrium 2, the fluid and/or gas may be passed from the first balloon 902 to the second balloon 904 and/or third balloon 914. Similarly, as pressure in the left atrium 2 drops, fluid and/or gas form the second balloon 904 and/or third balloon 914 may be pressed into the tube 905 and/or tubes 907 and/or back into the first balloon 902.

The second balloon 904 may be configured for placement in the IVC 10 and/or the third balloon 914 may be configured for placement within a superior vena cava (SVC) 22 of the heart 1. Thus, the second balloon 904 may be configured to obstruct blood flow upwards into the right atrium 5 and/or the third balloon 914 may be configured to obstruct blood flow downwards into the right atrium 5.

While the second balloon 904 and the third balloon 914 are shown inflated and/or expanded in FIG. 9, The second balloon 904 and/or third balloon 914 may be biased towards a deflated form. For example, the first balloon 902 may be biased towards an inflated form such that the first balloon 902 may be configured to maintain an inflated and/or expanded form in response to normal and/or relatively low pressure within the left atrium 2. In response to increased and/or raised blood pressure in the left atrium 2, the first balloon 902 may be configured to compress and/or deflate such that fluid and/or gas in the first balloon 902 is dispersed via the tube 905 and/or tubes 907 into the second balloon 904 and/or third balloon 914. As a result, the second balloon 904 and/or third balloon 914 may be configured inflate and/or expand as the pressure in the left atrium 2 increases. As the left atrium 2 pressure decreases and/or returns to normal levels, the second balloon 904 and/or third balloon 914 may be configured to compress and/or deflate and/or the first balloon 902 may be configured to reinflate.

The second balloon 904 and the third balloon 914 may be configured to inflate and/or deflate simultaneously and/or near simultaneously. For example, the second balloon 904 and the third balloon 914 may be configured to have generally equal responses to changes in left atrial pressure. The second balloon 904 and the third balloon 914 may have generally equal and/or different sizes. For example, the second balloon 904 may be configured to expand to fill the IVC 10 and/or the third balloon 914 may be configured to expand to fill the SVC 22. Depending on the sizes of the IVC 10 and SVC 22, the second balloon 904 and the third balloon 914 may have different expanded sizes and/or form and/or may be configured to fill in different amounts of time.

The system may comprise one or more anchors 906 configured to anchor the first balloon 902 in place. The one or more anchors 906 may have a shape and/or size configured to prevent the one or more anchors 906 from passing through an opening in the septum 18.

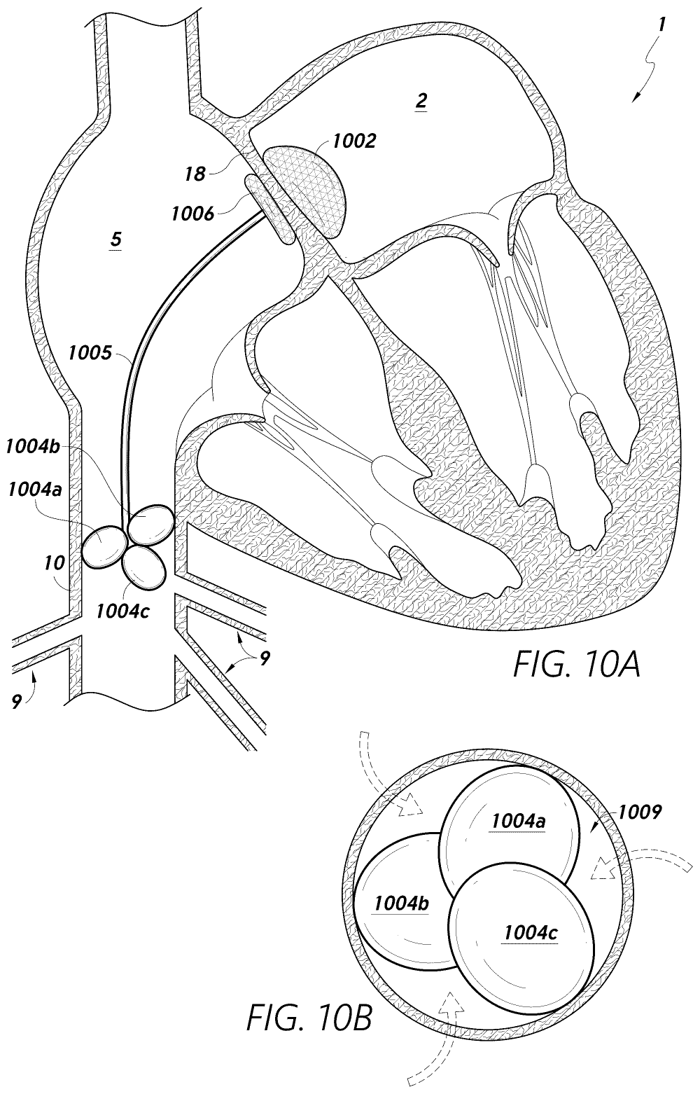

FIGS. 10A and 10B illustrate another modulation system implanted in a heart 1 and comprising one or more balloons in the IVC 10 in accordance with one or more examples. FIG. 10A provides a side view of the system and FIG. 10B provides an overhead view of one or more caval balloons 1004 disposed in the IVC 10. The modulation system may be pressure-induced and/or may comprise a first balloon 1002 disposed in the left atrium 2 and one or more caval balloons 1004, which can include a first caval balloon 1004a, a second caval balloon 1004b, and/or a third caval balloon 1004c, configured to obstruct blood flow into the right atrium 5. The first balloon 1002 and the caval balloons 1004 may be interconnected via a tube 1005 and/or via one or more branching tubes extending from a main tube 1005. The tube 1005 may include any suitable device configured for conveying liquid and/or gas between the balloons. In some examples, the tube 1005 may have generally thin and/or elongate tubular forms. The tube 1005 may provide a fluidic connection and/or channel between the balloons. For example, as a fluid and/or gas is pressed out of the first balloon 1002 (e.g., in response to increased pressure in the left atrium 2, the fluid and/or gas may be passed from the first balloon 1002 to the caval balloons 1004. Similarly, as pressure in the left atrium 2 drops, fluid and/or gas form the caval balloons 1004 may be pressed into the tube 1005 and/or back into the first balloon 1002.

In some examples, the caval balloons 1004 may form a cluster of balloons and/or may at least partially contact each other and/or at least partially overlap. As shown in FIG. 10B, while inflated, the caval balloons 1004 may be configured to form one or more gaps 1009 through which some minimal blood flow through the IVC 10 can pass.

The system may comprise one or more anchors 1006 configured to anchor the first balloon 1002 in place. The one or more anchors 1006 may have a shape and/or size configured to prevent the one or more anchors 1006 from passing through an opening in the septum 18.

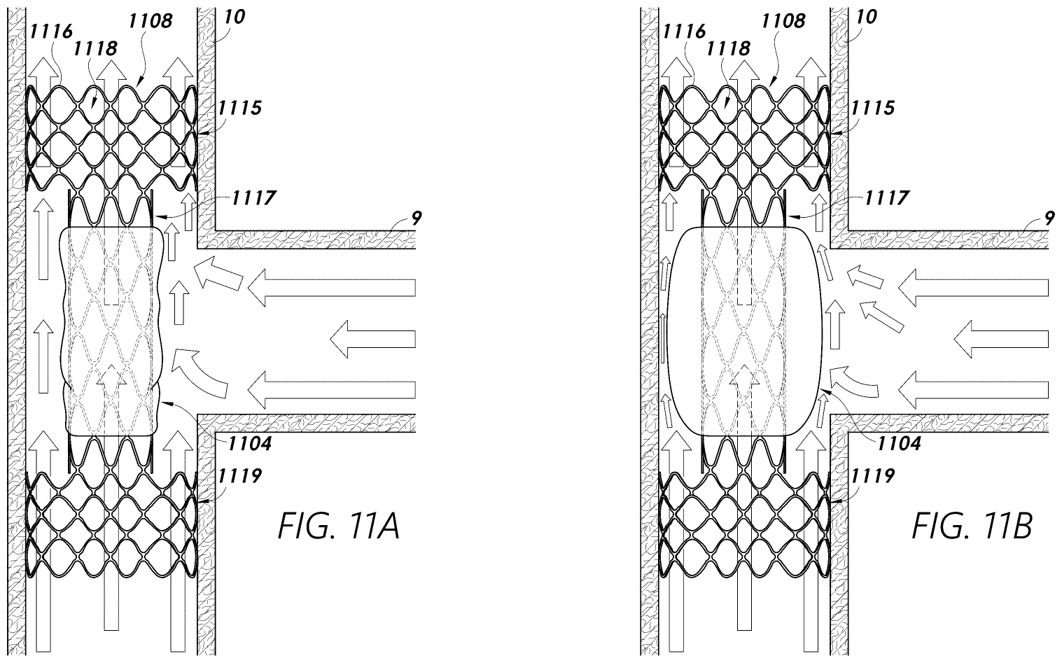

FIGS. 11A and 11B illustrate an example flow-regulating implant deployed at least partially within an IVC 10 in accordance with one or more embodiments. The implant may comprise a stent 1108 and/or frame configured to hold one or more balloons 1104. The stent 1108 and/or frame may have a generally tubular and/or cylindrical shape and/or may be at least partially compressible and/or expandable. The implant may be configured to allow longitudinal blood flow (e.g., towards the top of the page in FIGS. 11A and 11B) through the implant and/or lateral blood flow (e.g., towards the left side of the page in FIGS. 11A and 11B) through the sides of the implant (e.g., through the stent 1108 and/or frame).

In some embodiments, the implant may be configured to extend at least partially across one or more junctions between the IVC 10 and one or more hepatic veins 9. For example, when the balloon 1104 is inflated as shown in FIG. 11B, blood flow from one or more hepatic veins 9 may be prevented from entering the IVC 10 by the balloon 1104. However, some limited amount of blood flow from the one or more hepatic veins 9 may be enabled to pass through gaps around the balloon 1104 and/or to enter the IVC 10 and/or a lumen of the stent 1108. Once inside the IVC 10 and/or the lumen of the implant, the blood may be carried upward along the IVC 10 with the natural upward blood flow of the IVC.

The position of the implant may be adjusted following deployment of the implant within the IVC 10. For example, the implant may be configured such that a length of the implant exceeds a width of a hepatic vein 9 and/or such that a midsection 1117 of the stent 1108 extends across the hepatic vein 9. Thus, the implant may be adjusted upward and/or downward within the IVC 10 while maintaining complete and/or partial coverage of the hepatic vein 9. The implant may be configured for delivery via the IVC 10 and/or may be deployed at a position such that the implant simultaneously obstructs multiple hepatic veins 9.

In some embodiments, the stent 1108 may be configured to be expanded using a balloon expander and/or other means. The implant may be configured to be retrievable following deployment within the IVC 10. In some embodiments, an impact on blood flow of the implant may be evaluated while the implant is tethered to a delivery system. If the blood flow impact is less than or more than desired, the implant may be retrieved and/or adjusted. Upon determination that a desired blood flow impact has been reached, any tethering between the delivery systems and the implant may be removed.

The implant may have any suitable size to allow the implant to fit within the IVC 10. In some cases, differently sized implants may be used depending on a determination of a size of a patient's IVC 10.

FIGS. 11A and 11B provide side views of the stent 1108 within the IVC 10 and/or along a major axis of the stent 1108. In some examples, the stent 1108 may comprise a network of one or more struts 1116 which may interconnect and/or form one or more cells 1118 between the struts 1116. One or more cells 1118 formed by the struts 1116 may have a generally diamond-shaped and/or rectangular form.

The stent 1108 may comprise a middle portion 1117 (e.g., midsection) situated between a first end portion 1115 and a second end portion 1119 of the stent 1108. The middle portion 1117, first end portion 1115, and/or second end portion 1119 may comprise generally circular and/or ovular rings. The first end portion 1115 and/or the second end portion 1119 may have greater diameters than the middle portion 1117.

The struts 1116 may be configured to bend and/or navigate with respect to each other to provide flexibility to the stent 1108. For example, the struts 1116 may be configured to bend to allow the stent 1108 to assume a generally compressed form within a catheter and/or other delivery device. Upon removal from the catheter and/or other delivery device, the stent 1108 and/or struts 1116 may be configured to relax and/or assume a default expanded and/or relaxed form.

The balloon 1104 may be disposed around an outer surface of the middle portion 1117 and/or may be disposed within a lumen formed by the middle portion 1117. The middle portion 1117 may be configured to hold the balloon 1104 in the deflated form shown in FIG. 11A at a distance from an inflow junction of a hepatic vein 9. For example, when the balloon 1104 is in a deflated and/or compressed form, the balloon 1104 may not contact and/or extend over an inflow junction of the hepatic vein 9. As shown in FIG. 11B, as the balloon inflates, the balloon 1104 may extend towards the inflow junction of the hepatic vein 9 and/or may more fully obstruct blood flow from the hepatic vein 9. While only a single hepatic vein 9 is shown in FIGS. 11A and 11B, the balloon 1104 may be configured to obstruct multiple hepatic veins 9.

The balloon 1104 may be attached to and/or may extend into one or more tubes (not shown) which may interconnect the balloon 1104 to one or more additional balloons. For example, the balloon 1104 may be fluidly connected to a balloon disposed in the left atrium (not shown). Accordingly, the balloon 1104 may be configured to selectively obstruct the IVC 10 and/or hepatic veins 9 in response to increased pressure in the left atrium.

In some examples, the balloon 1104 may have a ring-shaped and/or donut-shaped form, in which the balloon 1104 may have a circular and/or ovular shape around an interior lumen.

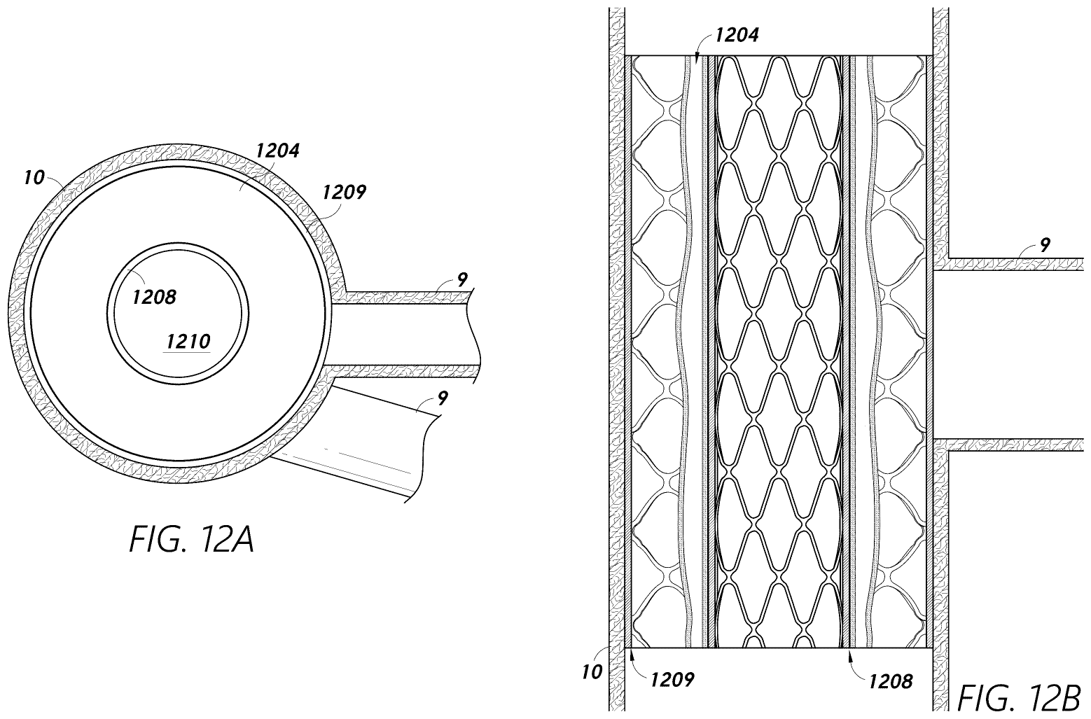

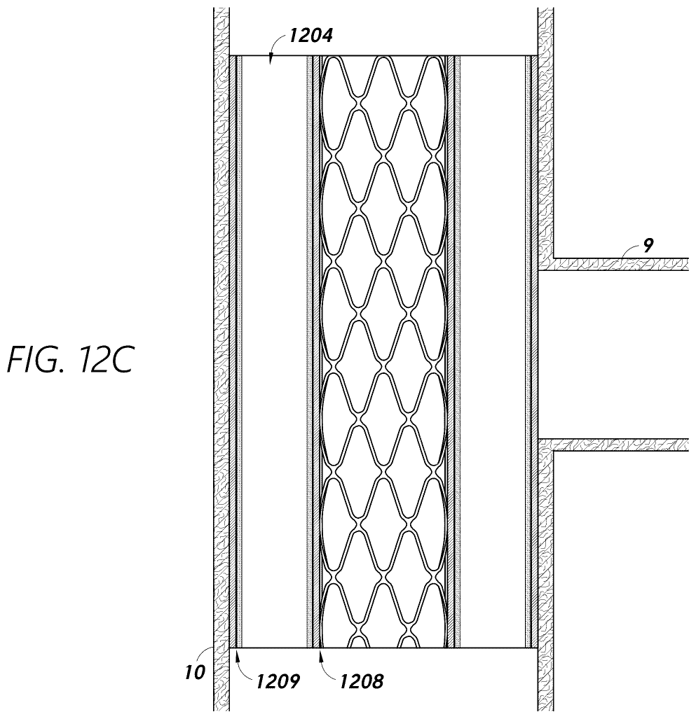

FIGS. 12A-12C illustrate another example implant comprising a caval balloon 1204 for placement in an IVC 10 and/or other blood vessel in accordance with one or more examples. The balloon 1204 and/or implant may comprise part of a modulation system configured to selectively modulate blood flow through the IVC 10, SVC, and/or other blood vessels in response to increased left atrial pressure.

FIG. 12A provides an overhead view of the implant with the balloon 1204 in an inflated and/or expanded form. FIG. 12B provides a side view of the implant with the balloon 1204 in a deflated and/or compressed form. FIG. 12C provides a side view of the implant with the balloon 1204 in an inflated and/or expanded form. The implant may comprise a caval balloon 1204 disposed and/or sandwiched between an inner frame 1208 (e.g., an inner stent) and/or an outer frame 1209 (e.g., an outer stent). The inner frame 1208 and/or the outer frame 1209 may have generally cylindrical and/or tubular forms and/or may be configured to fully and/or at least partially enclose the balloon 1204. In some examples, the balloon 1204 may similarly have a ring-shaped and/or donut-shaped form, in which the balloon 1204 may have a circular and/or ovular shape around an interior lumen 1210.

The balloon 1204 may be attached to and/or may extend into one or more tubes (not shown) which may interconnect the balloon 1204 to one or more additional balloons. For example, the balloon 1204 may be fluidly connected to a balloon disposed in the left atrium (not shown). Accordingly, the balloon 1204 may be configured to selectively obstruct the IVC 10 and/or hepatic veins 9 in response to increased pressure in the left atrium.

In some examples, the balloon 1204 may be coupled and/or anchored to at least the inner frame 1208. As left atrial pressure increases, additional gas and/or fluid may be driven into the balloon 1204 such that the balloon 1204 may at least partially inflate and/or expand. As a result, the balloon 1204 may press against the outer frame 1209 and/or may press the outer frame 1209 outwardly and/or into contact with walls of the IVC 10 and/or against inflow junctions of one or more hepatic veins 9. In some examples, the inner frame 1208 and/or outer frame 1209 may be generally porous (e.g., having a wire frame form) and/or may provide generally minimal resistance to blood flow. Alternatively, the inner frame 1208 and/or outer frame 1209 may comprise coverings and/or may have generally solid forms to effectively obstruct blood flow. For example, the balloon 1204 may be configured to press the outer frame 1209 against an inflow junction of a hepatic vein 9 such that the outer frame 1209 at least partially obstructs the inflow junction of the hepatic vein 9. The implant may be configured to provide minimal obstruction of the IVC 10.

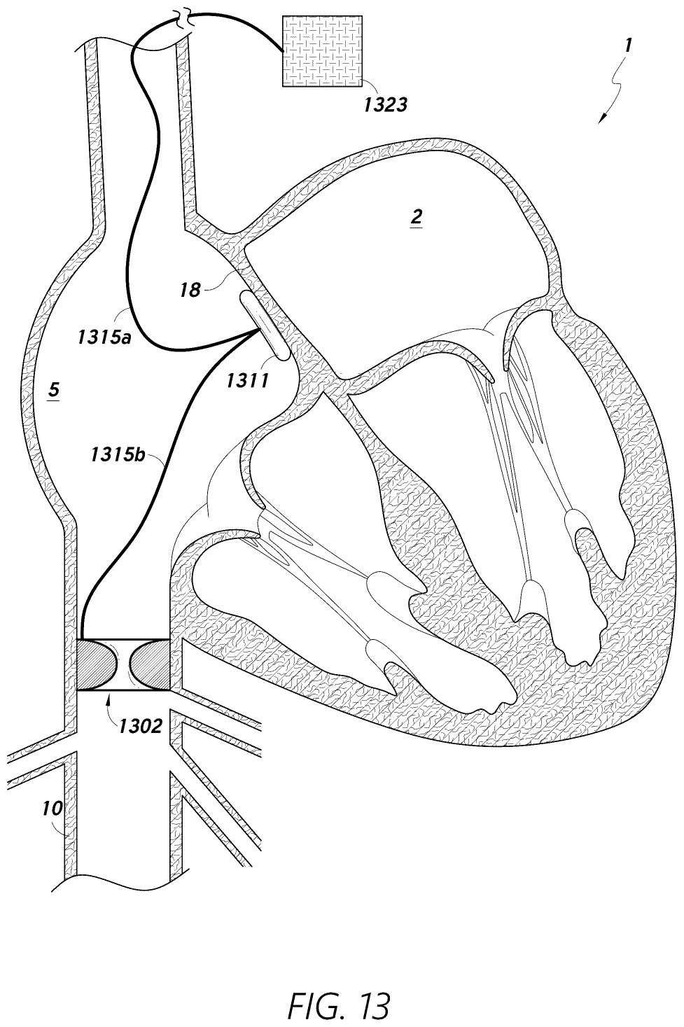

FIG. 13 illustrates another example modulation system comprising one or more sensors 1311 configured to control one or more means for obstructing, restricting, and/or modulation blood flow into the right atrium 5 in accordance with one or more examples of the present disclosure. In some examples, one or more pressure sensors 1311 may be anchored at least partially within the right atrium 5. One or more wires 1315 may extend from the sensor 1311, including a first wire 1315a and/or a second wire 1315b. The first wire 1315a may extend between the sensor 1311 and a generator 1323 (e.g., a subcutaneous generator) and/or the second wire 1315b may extend between the sensor 1311 and an obstruction element 1302, which can include one or more balloons and/or mechanical elements (e.g., petals, arms, pads, leaflets, etc.). The obstruction element 1302 can be disposed at least partially within the IVC 10.

The sensor 1311 may be configured to sense pressure increases within the right atrium 5 and/or left atrium 2. In response to detecting pressure changes, the sensor 1311 may be configured to transmit a signal and/or to send a current to the obstruction element 1302 to cause the obstruction element 1302 to adjust and/or move to increase an amount of obstruction of the IVC 10 to reduce blood flow into the right atrium 5. For example, movement of the obstruction element 1302 may close an orifice formed by the obstruction element 1302. In some examples, the obstruction element 1302 may form a ring with an orifice through a center portion of the obstruction element 1302.

In some examples, the obstruction element 1302 may be at least partially composed of Nitinol and/or other shape memory alloys and/or materials configured to alter and/or adjust in response to an electric and/or thermal stimulus. In another example, the obstruction element 1302 can comprise carbon nanotubes configured to move and/or adjust in response to an electric and/or thermal stimulus.

The generator 1323 may be configured to provide power to the sensor 1311. For example, the generator 1323 may provide electric power to the sensor 1311 to allow the sensor 1311 to sense pressure changes and/or transmit electric currents.

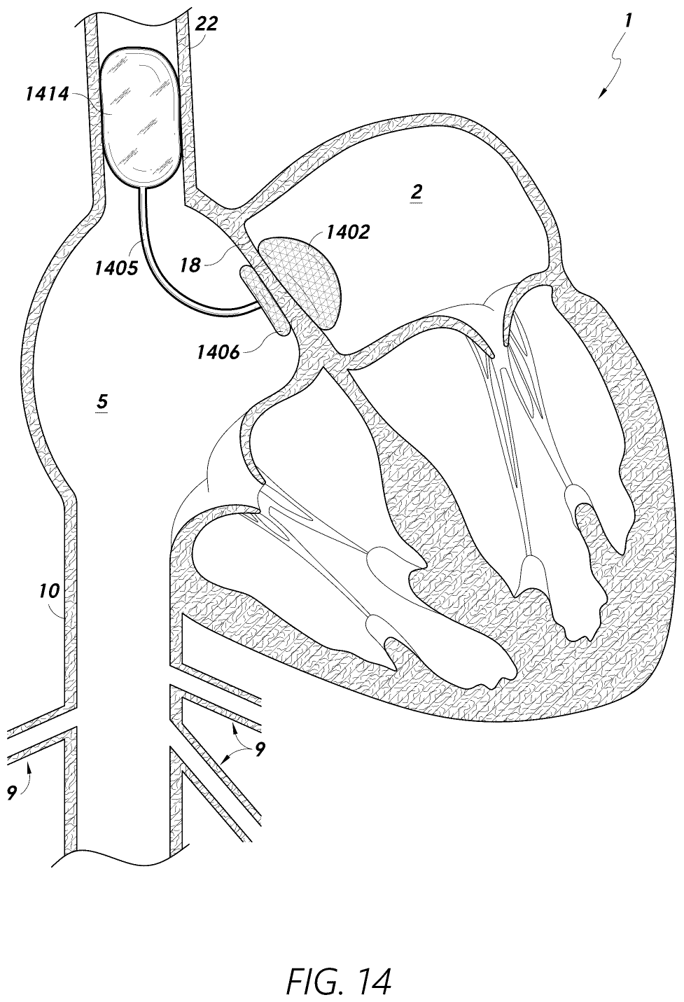

FIG. 14 illustrates an example pressure-induced modulation system comprising a first balloon 1402 disposed in the left atrium 2 and a second balloon 1414 disposed in the SVC 22 in accordance with one or more examples. The first balloon 1402 and the second balloon 1414 may be interconnected via a tube 1405, which may include any suitable device configured for conveying liquid and/or gas between the first balloon 1402 and the second balloon 1414. In some examples, the tube 1405 may have a generally thin and/or elongate tubular form. The tube 1405 may provide a fluidic connection and/or channel between the first balloon 1402 and the second balloon 1414. For example, as a fluid and/or gas is pressed out of the first balloon 1402, the fluid and/or gas may be passed from the first balloon 1402 to the second balloon 1414, and vice versa.

In some examples, the tube 1405 may connect to and/or extend from the first balloon 1402 and the second balloon 1414 via an open channel and/or a selectively closed channel. For example, one or more flaps may be used to selectively allow flow from the first balloon 1402 and/or the second balloon 1414 into the tube 1405 and/or vice versa. In some examples, the tube 1405 may be configured to be disposed at least partially within the right atrium 5 and/or SVC 22.

In some examples, the first balloon 1402 can have a dome and/or partial spherical (e.g., hemispherical) shape. For example, the first balloon 1402 may comprise half of a sphere and/or may have a generally flat base portion configured to rest against a surface of the atrial septum 18. As the first balloon 1402 extends away from the septum 18, the shape of the first balloon 1402 may become more rounded. However, the first balloon 1402 may have any suitable shape. For example, the first balloon 1402 may alternatively comprise a generally circular and/or ovular shape.

The first balloon 1402 may be secured to the septum 18 in any suitable manner. In some examples, a support anchor 1406 may be configured to anchor the first balloon 1402 in place. The anchor 1406 may comprise a generally solid and/or hollow structure configured to be disposed opposite the first balloon 1402 in the right atrium 5 and/or against the septum 18 on the right atrium 5 side. At least a portion of the septum 18 may be sandwiched between the anchor 1406 and the first balloon 1402. In some examples, the anchor 1406 may comprise an inflatable balloon and/or other expandable device. The anchor 1406 may be connected to the first balloon 1402 and/or may be an extension of the first balloon 1402. Alternatively, the anchor 1406 may be a separate device and/or may be joined to the first balloon 1402 via the tube 1405 and/or other tethering device. In some examples, the anchor 1406 may comprise a disc and/or similar device and/or may have a generally circular and/or ring-shaped form.

In some examples, the tube 1405 may be configured to extend at least partially through the anchor 1406 and/or through the septum 18 as the tube 1405 extends from the first balloon 1402 towards the SVC 22. The anchor 1406 and/or first balloon 1402 may comprise apertures and/or openings configured to receive and/or accommodate the tube 1405.

The second balloon 1414 may be delivered into the heart 1 separately and/or together with the first balloon 1402. In some examples, the second balloon 1414 may have a generally spherical form and/or may be configured to approximate a cross-sectional shape of the SVC 22. The tube 1405 may have a suitable length such that the second balloon 1414 may be configured to extend at least partially into the SVC 22 and/or in front of inflow channels from one or more hepatic veins 9. In some examples, the second balloon 1414 may be configured to inflate to a diameter that is greater than the one or more hepatic veins 9 and/or approximately equal to or greater than a diameter of the SVC 22.

The first balloon 1402, second balloon 1414, and/or anchor 1406 may have any suitable structure. In some examples, the first balloon 1402, second balloon 1414, and/or anchor 1406 may comprise an outer layer and/or an inner layer. The inner layer may comprise a generally flexible, stretchy, and/or elastic material, which can include rubber, latex, and/or similar materials. The outer layer may have a generally rigid and/or bendable structure and/or may comprise one or more interwoven wires and/or lines of material. The outer layer may be at least partially composed of Nitinol and/or other shape memory alloys.

The first balloon 1402 and/or second balloon 1414 may be configured to be filled with an incompressible fluid. In some examples, the first balloon 1402 and/or second balloon 1414 may be implanted through an endovascular transeptal approach.

In some examples, the first balloon 1402 and/or the second balloon 1414 may comprise various features configured to regulate inflation and/or deflation and/or to regulate a rate of inflation and/or deflation of the first balloon 1402 and/or the second balloon 1414. For example, one or more springs, coils, and/or similar elements (e.g., Nitinol wire forms) may be attached to an exterior of the first balloon 1402 and/or second balloon 1414 and/or may be placed inside the first balloon 1402 and/or the second balloon 1414 to regulate the rate of deflation and/or inflation.

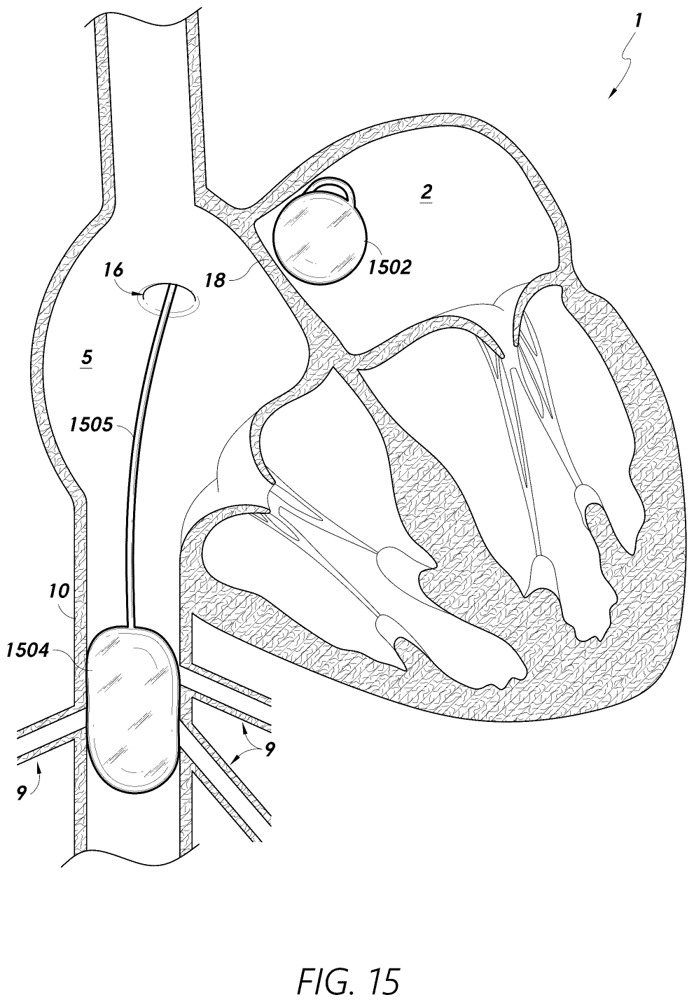

FIG. 15 illustrates an example pressure-induced modulation system comprising a first balloon 1502 disposed in the left atrium 2 and a second balloon 1504 disposed in the IVC 10 in accordance with one or more examples. The first balloon 1502 and the second balloon 1504 may be interconnected via a tube 1505, which may include any suitable device configured for conveying liquid and/or gas between the first balloon 1502 and the second balloon 1504. In some examples, the tube 1505 may have a generally thin and/or elongate tubular form. The tube 1505 may provide a fluidic connection and/or channel between the first balloon 1502 and the second balloon 1504. For example, as a fluid and/or gas is pressed out of the first balloon 1502, the fluid and/or gas may be passed from the first balloon 1502 to the second balloon 1504, and vice versa.

In some examples, the tube 1505 may connect to and/or extend from the first balloon 1502 and the second balloon 1504 via an open channel and/or a selectively closed channel. For example, one or more flaps may be used to selectively allow flow from the first balloon 1502 and/or the second balloon 1504 into the tube 1505 and/or vice versa. In some examples, the tube 1505 may be configured to be disposed at least partially within the right atrium 5 and/or IVC 10 and/or may be configured to extend through the coronary sinus 16 to extend from the right atrium 5 to the left atrium 2.

In some examples, the first balloon 1502 can have a spherical and/or other shape. The tube 1505 may be configured to enter the left atrium 2 via the coronary sinus 16 and/or one or more branching blood vessels. The first balloon 1502 may be configured to extend into the left atrium 2 and/or away from a junction between the left atrium 2 and the coronary sinus 16 and/or branching blood vessels to avoid blockage of the coronary sinus 16. In some examples, the first balloon 1502 may be placed in the left atrium 2 with a stent, anchor and/or similar device configured to hold the first balloon 1502 distally from the coronary sinus 16 and/or to maintain a flow pathway between the left atrium 2 and the coronary sinus 16. The first balloon 1502 may be anchored to one or more walls of the left atrium 2, which can include the septum 18.

The second balloon 1504 may be delivered into the heart 1 separately and/or together with the first balloon 1502. In some examples, the second balloon 1504 may have a generally spherical form and/or may be configured to approximate a cross-sectional shape of the IVC 10. The tube 1505 may have a suitable length such that the second balloon 1504 may be configured to extend at least partially into the IVC 10 and/or in front of inflow channels from one or more hepatic veins 9. In some examples, the second balloon 1504 may be configured to inflate to a diameter that is greater than the one or more hepatic veins 9 and/or approximately equal to or greater than a diameter of the IVC 10.

The first balloon 1502 and/or second balloon 1504 may have any suitable structure. In some examples, the first balloon 1502 and/or second balloon 1504 may comprise an outer layer and/or an inner layer. The inner layer may comprise a generally flexible, stretchy, and/or elastic material, which can include rubber, latex, and/or similar materials. The outer layer may have a generally rigid and/or bendable structure and/or may comprise one or more interwoven wires and/or lines of material. The outer layer may be at least partially composed of Nitinol and/or other shape memory alloys.

The first balloon 1502 and/or second balloon 1504 may be configured to be filled with an incompressible fluid. In some examples, the first balloon 1502 and/or second balloon 1504 may be implanted through an endovascular transeptal approach.

In some examples, the first balloon 1502 and/or the second balloon 1504 may comprise various features configured to regulate inflation and/or deflation and/or to regulate a rate of inflation and/or deflation of the first balloon 1502 and/or the second balloon 1504. For example, one or more springs, coils, and/or similar elements (e.g., Nitinol wire forms) may be attached to an exterior of the first balloon 1502 and/or second balloon 1504 and/or may be placed inside the first balloon 1502 and/or the second balloon 1504 to regulate the rate of deflation and/or inflation.

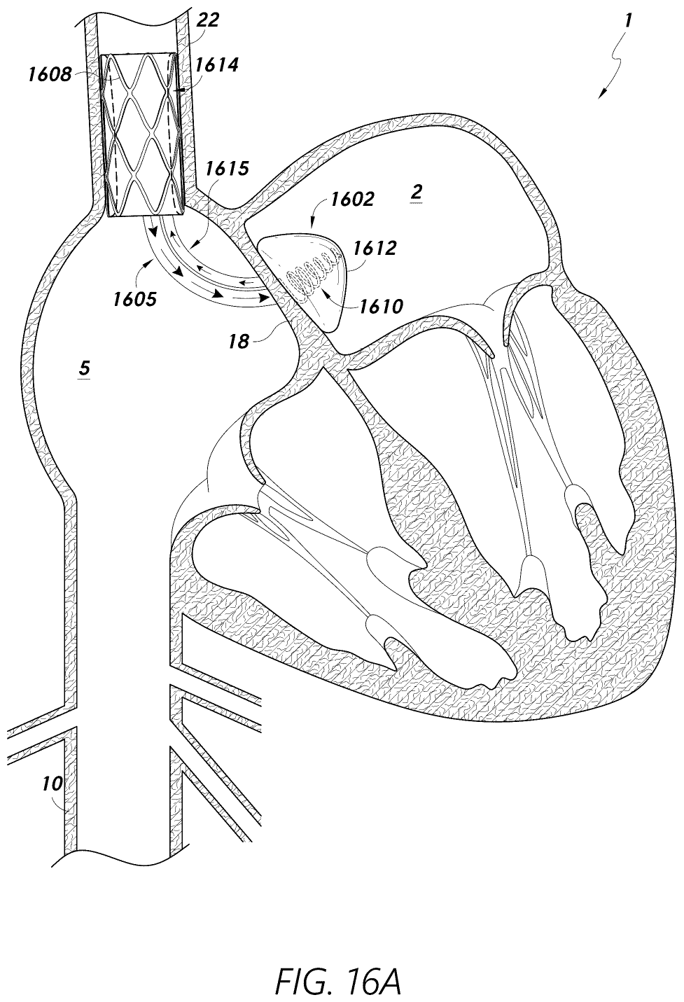

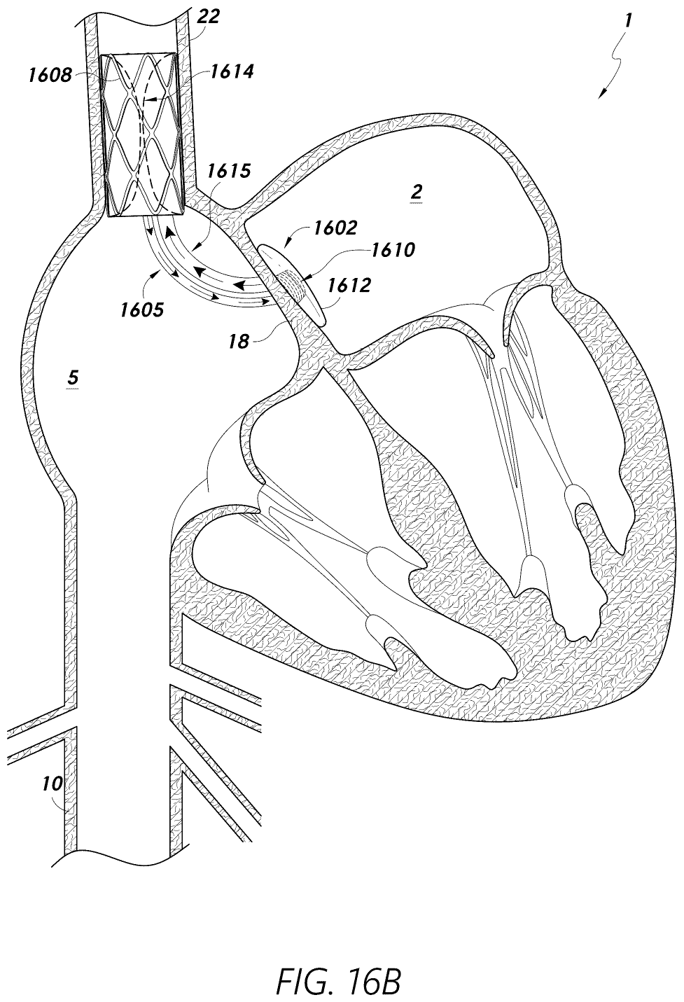

FIGS. 16A and 16B illustrate an example pressure-induced modulation system comprising a first balloon 1602 disposed in the left atrium 2 and a second balloon 1614 disposed in the SVC 22 in accordance with one or more examples. The first balloon 1602 and the second balloon 1614 may be interconnected via one or more tubes, which can include a first tube 1605 and/or a second tube 1615, which may include any suitable device configured for conveying liquid and/or gas between the first balloon 1602 and the second balloon 1614. In some examples, the first tube 1605 may be configured to convey fluid from the second balloon 1614 to the first balloon 1602 and/or the second tube 1615 may be configured to convey fluid from the first balloon 1602 to the second balloon 1614.

The first tube 1605 and/or second tube 1615 may provide a fluidic connection and/or channel between the first balloon 1602 and the second balloon 1614. For example, as a fluid and/or gas is pressed out of the first balloon 1602, the fluid and/or gas may be passed from the first balloon 1602 to the second balloon 1614 (e.g., via the second tube 1615), and vice versa.

In some examples, the first tube 1605 and/or second tube 1615 may connect to and/or extend from the first balloon 1602 and the second balloon 1614 via an open channel and/or a selectively closed channel. For example, one or more flaps may be used to selectively allow flow from the first balloon 1602 and/or the second balloon 1614 into the first tube 1605 and/or second tube 1615 and/or vice versa. In some examples, the first tube 1605 and/or second tube 1615 may be configured to be disposed at least partially within the right atrium 5 and/or SVC 22.

In some examples, the first balloon 1602 can have a dome and/or partial spherical (e.g., hemispherical) shape. For example, the first balloon 1602 may comprise half of a sphere and/or may have a generally flat base portion configured to rest against a surface of the atrial septum 18. As the first balloon 1602 extends away from the septum 18, the shape of the first balloon 1602 may become more rounded. However, the first balloon 1602 may have any suitable shape. For example, the first balloon 1602 may alternatively comprise a generally circular and/or ovular shape.

The first balloon 1602 may comprise a support device 1610 configured to facilitate expansion and/or deflation of the first balloon 1602. The support device 1610 can comprise a spring, coil, and/or similar mechanism. In some examples, the support device 1610 may be disposed at least partially within a membrane 1612 and/or covering of the first balloon 1602. The support device 1610 may extend at least partially within a hollow interior formed by the membrane 1612 and/or may be encased and/or enclosed by the membrane 1612. For example, the support device 1610 may form a skeleton for the membrane 1612.

In some examples, the support device 1610 may have an internal bias. For example, the support device 1610 may be biased to the expanded form shown in FIG. 16A. As blood pressure increases in the left atrium 2 and the first balloon 1602 compresses, the support device 1610 may similarly compress to the form shown in FIG. 16B. However, as blood pressure in the left atrium 2 decreases, the internal bias of the support device 1610 may cause the support device 1610 to press the membrane 1612 of the first balloon 1602 outwardly and/or towards the expanded form of FIG. 16A. Thus, both fluid from the second balloon 1614 and the bias of the support device 1610 may together facilitate expansion of the first balloon 1602.

The second balloon 1614 may be delivered into the heart 1 separately and/or together with the first balloon 1602. In some examples, the second balloon 1614 may have a generally spherical form and/or may be configured to approximate a cross-sectional shape of the SVC 22. The first tube 1605 and/or second tube 1615 may have a suitable length such that the second balloon 1614 may be configured to extend at least partially into the SVC 22 and/or in front of inflow channels from one or more hepatic veins. The second balloon 1614 may be at least partially enclosed by a stent 1608 and/or similar device. The stent 1608 may be configured to hold the second balloon 1614 in place within the SVC 22.

In some examples, the first balloon 1602 and/or second balloon 1614 may comprise an outer layer and/or an inner layer. The inner layer may comprise a generally flexible, stretchy, and/or elastic material, which can include rubber, latex, and/or similar materials. The outer layer may have a generally rigid and/or bendable structure and/or may comprise one or more interwoven wires and/or lines of material. The outer layer may be at least partially composed of Nitinol and/or other shape memory alloys.

The first balloon 1602 and/or second balloon 1614 may be configured to be filled with an incompressible fluid. In some examples, the first balloon 1602 and/or second balloon 1614 may be implanted through an endovascular transeptal approach.

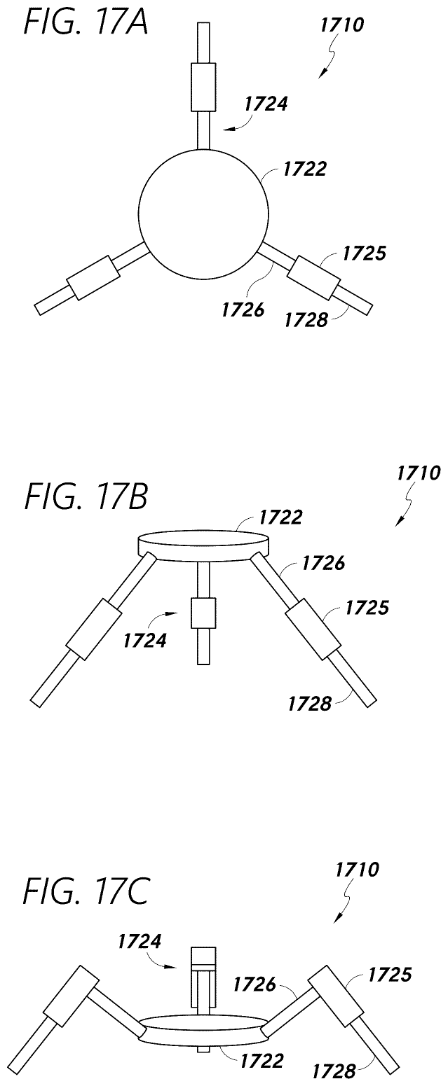

FIGS. 17A-17C illustrate an example support device 1710 in accordance with one or more examples. FIG. 17A provides a top view of the support device 1710 in an expanded form, FIG. 17B provides a side view of the support device 1710 in the expanded form, and FIG. 17C provides a side view of the support device 1710 in a compressed form. The support device 1710 may be configured to expand and/or compress passively and/or actively. In some examples, the support device 1710 may be configured to compress to the form shown in FIG. 17C in response to increased external pressure (e.g., increased left atrial pressure). Similarly, the support device 1710 may be configured to expand to the form shown in FIGS. 17A and 17B in response to a decrease in external pressure (e.g., decreased left atrial pressure).

The support device 1710 can have any of a variety of forms. In the example shown in FIGS. 17A-17C, the support device 1710 comprises a circular platform 1722 at a central point of the support device 1710. The platform 1722 may be disposed at or near proximal ends of one or more legs 1724 of the device 1710. The legs 1724 may comprise joints 1725 configured to allow bending and/or angular adjustment of proximal portions 1726 of the legs 1724 relative to distal portions 1728 of the legs 1724, as shown in FIG. 17C. The platform 1722 may be configured to be raised and/or lowered in response to bending and/or angular adjustment of the legs 1724. For example, when the legs 1724 have a generally straight configuration (as shown in FIGS. 17A and 17B), the platform 1722 may have a raised configuration and/or when the legs 1724 have a bent configuration (as shown in FIG. 17C), the platform 1722 may have a lowered configuration. The platform 1722 may be configured to provide a landing pad for pressure from blood pressure around the device 1710 and/or balloon.

While FIGS. 17A-17C illustrate the device 1710 comprising three legs 1724, the device 1710 may comprise any number of legs (e.g., two, four, etc.). Moreover, while the platform 1722 is shown having a circular shape, the platform may have any suitable shape and/or size. The legs 1724 may be configured to extend at an approximately 45-degree angle from the platform 1722, such that the legs 1724 extend laterally and longitudinally away from the platform 1722.

In some examples, the device 1710 may be biased to the expanded configuration and/or may move to the compressed configuration only in response to increased external pressure (e.g., increased left atrial pressure). The device 1710 may be configured to move to the compressed configuration only in response to a threshold amount of external pressure. For example, the device 1710 may be configured to resist moving to the compressed configuration until external pressure exceeds a threshold amount. The transition from the expanded configuration to the compressed configuration and/or from the compressed configuration to the expanded configuration may be quick and/or may be completed in a single motion. In some examples, the device 1710 may comprise one or more features configured to increase a resistance in configuration changes of the device 1710. For example, the device 1710 may comprise one or more tabs, dimples, cavities, notches, pegs, and/or similar mechanisms configured to hold the device in the expanded and/or compressed configuration. In one or example, the joint 1725 may comprise one or more raised bumps configured aligned with sides of the proximal portions 1726 of the legs 1724 to provide some resistance to movement of the proximal portions 1726 relative to the joint 1725 and/or distal portions 1726.

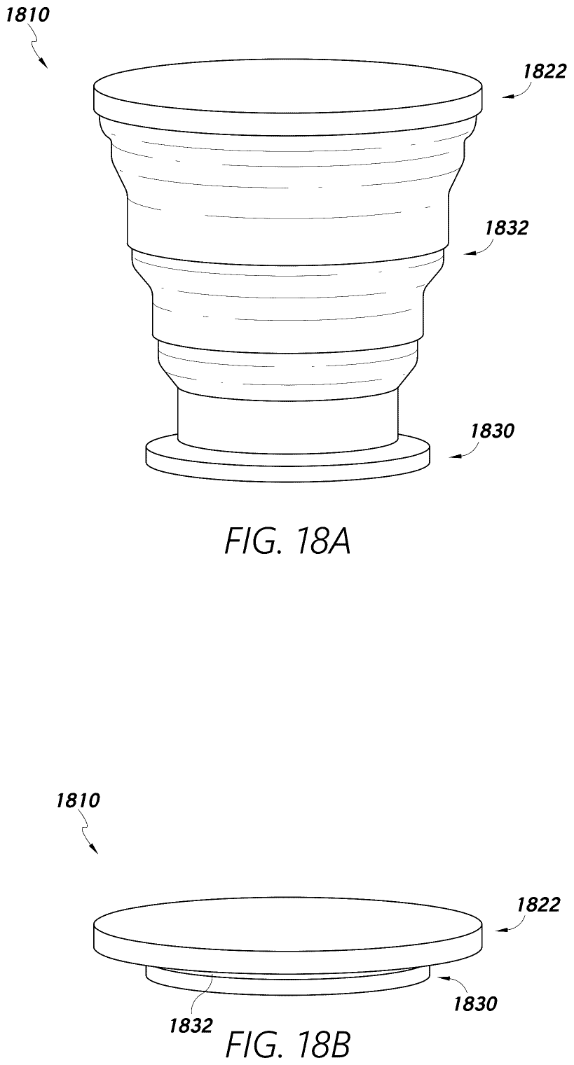

FIGS. 18A and 18B illustrate another example support device 1810 in accordance with one or more examples. FIG. 18A illustrates the support device 1810 in an expanded configuration and FIG. 18B illustrates the support device 1810 in a compressed configuration. The device 1810 can comprise a platform 1822 and/or a base 1830. A position of the platform 1822 relative to the base 1830 may be managed via a flexible midsection 1832. The midsection 1832 may be configured to form folds and/or bends. In some examples, the midsection 1832 may be biased towards an unfolded and/or generally straight form in which the midsection 1832 holds the platform 1822 at a maximum distance from the base 1830. In response to external pressure, the midsection 1832 may be configured to fold and/or bend to allow the platform 1822 to move towards the base 1830, as shown in FIG. 18B. Similarly, in response to reduced external pressure, the midsection 1832 may be configured to straighten and/or expand to press the platform 1822 away from the base 1830.

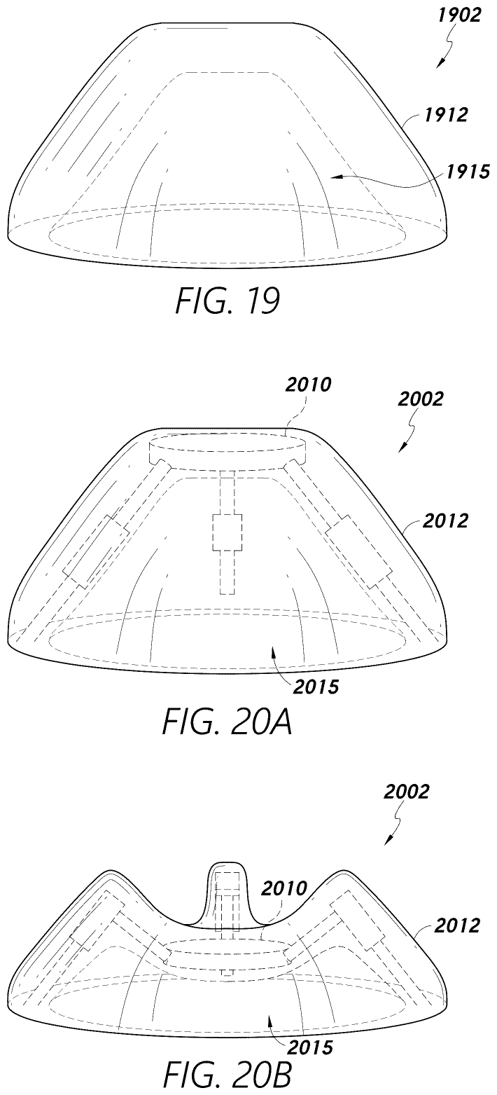

FIG. 19 illustrates an example balloon 1902 configured for use with one or more support devices (see, e.g., FIG. 20) in accordance with one or more examples. The balloon 1902 may comprise a membrane 1912 extending around a generally hollow interior 1915. The membrane 1912 may have a thickness and/or may be configured to encase and/or hold one or more support devices.

FIGS. 20A and 20B illustrate an example balloon 2002 configured for use with one or more support devices 2010 in accordance with one or more examples. FIG. 20A illustrates the balloon 2002 in an expanded configuration and FIG. 20B illustrates the balloon 2002 in a compressed configuration. The balloon 2002 may comprise a membrane 2012 extending around a generally hollow interior 2015. The membrane 2012 may have a thickness and/or may be configured to encase and/or hold one or more support devices 2010.

The balloon 2002 may be configured to transition from the expanded configuration to the compressed configuration and/or from the compressed configuration to the expanded configuration. In some examples, the balloon 2002 and/or device 2010 may be configured to transition in a single pulse in response to external pressure rising above and/or falling below a threshold value. A threshold pressure level required to cause the balloon 2002 to transition from the expanded configuration to the collapsed configuration may be different than the threshold pressure level required to cause the balloon 2002 to transition from the collapsed configuration to the expanded configuration. For example, the balloon 2002 may be configured to transition from the expanded configuration to the collapsed configuration only once external pressure exceeds a first pressure level. However, the balloon 2002 may not transition back from the collapsed configuration to the expanded configuration in response to external pressure falling below the first pressure level. Rather, the balloon 2002 may be configured to transition back from the collapsed configuration to the expanded configuration in response to external pressure falling below a second pressure level that is less than the first pressure level.

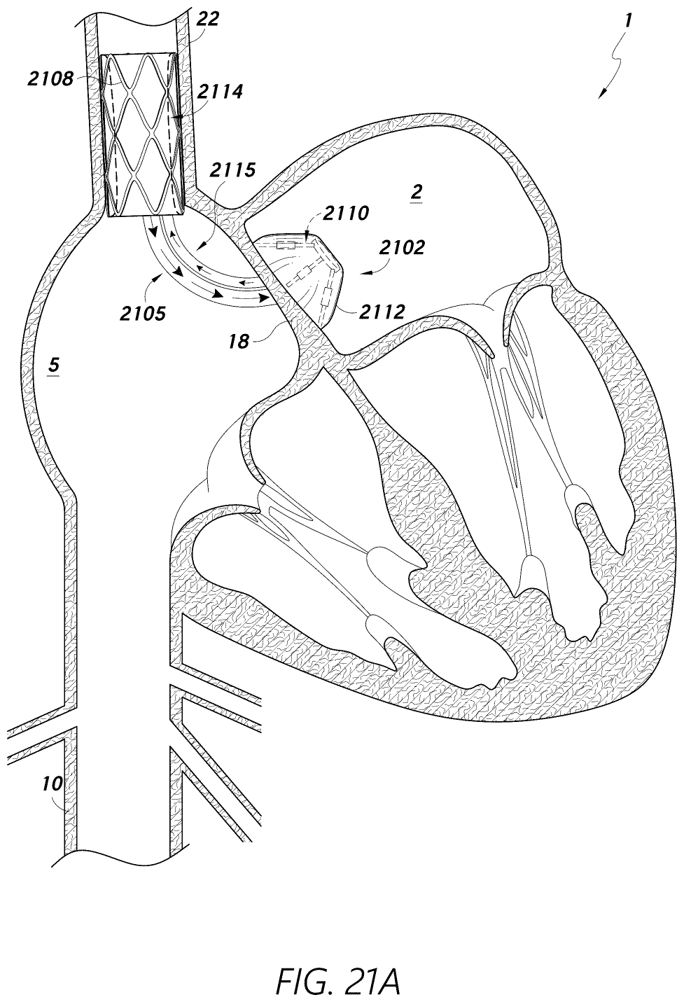

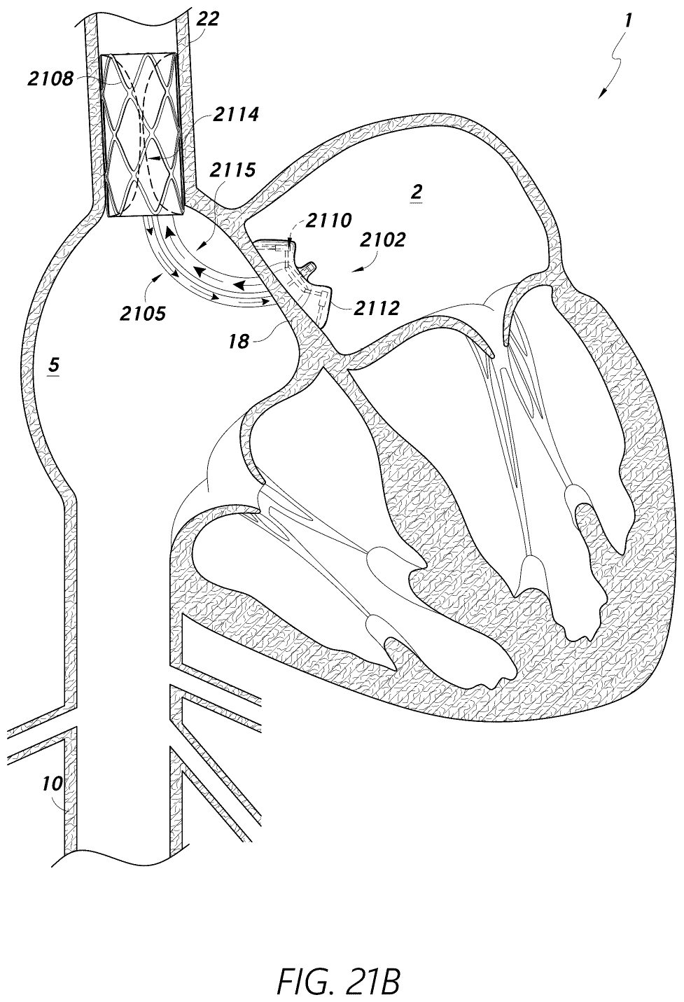

FIGS. 21A and 21B illustrate an example pressure-induced modulation system comprising a first balloon 2102 disposed in the left atrium 2 and a second balloon 2114 disposed in the SVC 22 in accordance with one or more examples. FIG. 21A illustrates a default configuration of the system in which the first balloon 2102 is in an inflated and/or expanded state and/or the second balloon 2114 is in a generally compressed and/or deflated state. FIG. 21B illustrates a configuration in which elevated pressure in the left atrium 2 causes deflation and/or compression of the first balloon 2102 and/or fluid passes from the first balloon 2102 to the second balloon 2114. The first balloon 2102 and the second balloon 2114 may be interconnected via one or more tubes, which can include a first tube 2105 and/or a second tube 2115. Suitable tubes can include devices configured for conveying liquid and/or gas between the first balloon 2102 and the second balloon 2114. In some examples, the first tube 2105 may be configured to convey fluid from the second balloon 2114 to the first balloon 2102 and/or the second tube 2115 may be configured to convey fluid from the first balloon 2102 to the second balloon 2114.

The first tube 2105 and/or second tube 2115 may provide a fluidic connection and/or channel between the first balloon 2102 and the second balloon 2114. For example, as a fluid and/or gas is pressed out of the first balloon 2102, the fluid and/or gas may be passed from the first balloon 2102 to the second balloon 2114 (e.g., via the second tube 2115), and vice versa.

In some examples, the first tube 2105 and/or second tube 2115 may connect to and/or extend from the first balloon 2102 and the second balloon 2114 via an open channel and/or a selectively closed channel. For example, one or more flaps may be used to selectively allow flow from the first balloon 2102 and/or the second balloon 2114 into the first tube 2105 and/or second tube 2115 and/or vice versa. In some examples, the first tube 2105 and/or second tube 2115 may be configured to be disposed at least partially within the right atrium 5 and/or SVC 22.

In some examples, the first balloon 2102 can have a dome and/or partial spherical (e.g., hemispherical) shape. For example, the first balloon 2102 may comprise half of a sphere and/or may have a generally flat base portion configured to rest against a surface of the atrial septum 18. As the first balloon 2102 extends away from the septum 18, the shape of the first balloon 2102 may become more rounded. However, the first balloon 2102 may have any suitable shape. For example, the first balloon 2102 may alternatively comprise a generally circular and/or ovular shape.

The first balloon 2102 may comprise a support device 2110 configured to facilitate expansion and/or deflation of the first balloon 2102. The support device 2110 can comprise a platform and/or one or more flexible and/or extendible mechanisms. For example, the device 2110 can comprise one or more legs configured to bend (e.g., at one or more joints). The device 2110 can additionally or alternatively comprise a generally flexible midsection extending between the platform and a base portion of the device 2110.

In some examples, the support device 2110 may be disposed at least partially within a membrane 2112 and/or covering of the first balloon 2102. The support device 2110 may extend at least partially within a hollow interior formed by the membrane 2112 and/or may be encased and/or enclosed by the membrane 2112. For example, the support device 2110 may form a skeleton for the membrane 2112. Movement of the device 2110 may be configured to cause corresponding movement of the first balloon 2102. For example, where the device 2110 is disposed within the hollow interior of the first balloon 2102 and/or not encased within the membrane 2112, the platform of the device 2110 may be attached to the membrane 2112 such that when the platform moves towards the septum 18, the membrane 2112 may be pulled with the device 2110.

In some examples, the support device 2110 may have an internal bias. For example, the support device 2110 may be biased to the expanded form shown in FIG. 21A. The expanded form may cause the first balloon 2102 to extend away from the atrial septum 18. As blood pressure increases in the left atrium 2 and the first balloon 2102 compresses, the support device 2110 may similarly compress (e.g., towards the atrial septum 18) to the form shown in FIG. 21B. However, as blood pressure in the left atrium 2 decreases, the internal bias of the support device 2110 may cause the support device 2110 to press the membrane 2112 of the first balloon 2102 outwardly and/or towards the expanded form of FIG. 21A. Thus, both fluid from the second balloon 2114 and the bias of the support device 2110 may together facilitate expansion of the first balloon 2102.

The first balloon 2102 may be configured to transition from the expanded configuration of FIG. 21A to the collapsed configuration of the FIG. 21B and/or vice versa in a single motion and/or in a rapid manner. In this way, the first balloon 2102 may be configured to cause a burst of fluid to pass from the first balloon 2102 to the second balloon 2114 and/or vice versa.

In some examples, the first balloon 2102 may comprise multiple devices 2110, with each device 2110 configured to transition between expanded and collapsed configurations. Where multiple devices 2110 are used, each device 2110 may be configured to transition between the expanded and collapsed configurations at different pressures, thereby creating a staggered transition of the first balloon 2102. Moreover, one or more devices 2110 may comprise multiple transition states. For example, a device 2110 may be configured to transition from an expanded configuration to a medium configuration and/or vice versa and/or from the medium configuration to a collapsed configuration and/or vice versa.

The first balloon 2102 may be secured to the septum 18 in any suitable manner. In some examples, a support anchor may be configured to anchor the first balloon 2102 in place.

The second balloon 2114 may be delivered into the heart 1 separately and/or together with the first balloon 2102. In some examples, the second balloon 2114 may have a generally spherical form and/or may be configured to approximate a cross-sectional shape of the SVC 22. The first tube 2105 and/or second tube 2115 may have a suitable length such that the second balloon 2114 may be configured to extend at least partially into the SVC 22 and/or in front of inflow channels from one or more hepatic veins 9. The second balloon 2114 may be at least partially enclosed by a stent 2108 and/or similar device. The stent 2108 may be configured to hold the second balloon 2114 in place within the SVC 22.

In some examples, the first balloon 2102 and/or second balloon 2114 may comprise an outer layer and/or an inner layer. The inner layer may comprise a generally flexible, stretchy, and/or elastic material, which can include rubber, latex, and/or similar materials. The outer layer may have a generally rigid and/or bendable structure and/or may comprise one or more interwoven wires and/or lines of material. The outer layer may be at least partially composed of Nitinol and/or other shape memory alloys.

The first balloon 2102 and/or second balloon 2114 may be configured to be filled with an incompressible fluid. In some examples, the first balloon 2102 and/or second balloon 2114 may be implanted through an endovascular transeptal approach.

Described herein are various example medical implants and/or delivery methods. Some examples described herein may be used in combination and/or may be used independently.
Example 1: A system comprising a first obstruction element disposed at least partially within an inferior vena cava (IVC), superior vena cava (SVC), or hepatic vein of a heart, the first obstruction element configured to expand and compress in response to pressure changes in the heart.
Example 2: The system of any example herein, in particular example 1, wherein the first obstruction element comprises a first balloon.
Example 3: The system of any example herein, in particular example 2, further comprising: a first tube; and a second balloon, wherein the first balloon is connected via the first tube to the second balloon disposed at least partially within a left atrium of the heart.
Example 4: The system of any example herein, in particular example 3, wherein the first tube is configured to convey a liquid or gas between the first balloon and the second balloon.
Example 5: The system of any example herein, in particular example 4, wherein the second balloon is configured to deflate in response to increased pressure in the left atrium and wherein the first balloon is configured to inflate in response to deflation of the second balloon.
Example 6: The system of any example herein, in particular example 4, wherein the second balloon is configured to maintain an inflated state in response to pressure in the left atrium being below a threshold amount, and wherein the first balloon is configured to maintain a deflated state while the second balloon is in the inflated state.
Example 7: The system of any example herein, in particular example 6, wherein the first balloon is disposed at least partially within the IVC.
Example 8: The system of any example herein, in particular example 6, wherein the first balloon is disposed at least partially within the SVC.
Example 9: The system of any example herein, in particular example 6, wherein the first tube is configured to extend at least partially through a coronary sinus of the heart between the first balloon and the second balloon.
Example 10: The system of any example herein, in particular example 3, further comprising an anchor disposed at least partially within a right atrium, wherein the second balloon is attached to the anchor.
Example 11: The system of any example herein, in particular example 10, wherein the anchor is a disc anchor.
Example 12: The system of any example herein, in particular example 3, wherein the second balloon has a hemispherical shape and comprises a generally flat base configured to extend along a septal wall of the left atrium.
Example 13: The system of any example herein, in particular example 2, further comprising a first stent disposed at least partially within the IVC.
Example 14: The system of any example herein, in particular example 13, wherein the first stent is configured to at least partially enclose the first balloon.
Example 15: The system of claim 13 or claim 14, wherein the first stent comprises a wire frame and wherein the first stent is configured to allow blood flow through the wire frame.
Example 16: The system of any example herein, in particular example 13, wherein the first stent has a cylindrical shape.
Example 17: The system of any example herein, in particular example 13, wherein the first balloon is attached to an outer surface of a midsection of the first stent.
Example 18: The system of any example herein, in particular example 17, wherein the first stent comprises a first end and a second end, and wherein a diameter of the midsection is less than diameters of the first end and second end.
Example 19: The system of any example herein, in particular example 17, wherein the first balloon has a ring shape and forms a central orifice.
Example 20: The system of any example herein, in particular example 19, further comprising a second stent at least partially enclosing the first balloon, wherein the first balloon is disposed between the first stent and the second stent.
Example 21: The system of any example herein, in particular example 3, wherein the first balloon is disposed at least partially within a first hepatic vein, the system further comprising a third balloon disposed in a second hepatic vein.
Example 22: The system of any example herein, in particular example 21, further comprising a second tube, wherein the third balloon is connected via the second tube to the second balloon.
Example 23: The system of any example herein, in particular example 22, wherein the first tube and the second tube branch from a main tube.
Example 24: The system of any example herein, in particular example 3, further comprising: a second tube; and a third balloon disposed at least partially within the SVC, wherein the third balloon is connected via the second tube to the second balloon, and wherein the first balloon is disposed at least partially within the IVC or the hepatic vein.
Example 25: The system of any example herein, in particular example 24, wherein the first tube and the second tube branch from a main tube.
Example 26: The system of any example herein, in particular example 3, further comprising a third balloon disposed in the IVC or hepatic veins, wherein the third balloon is connected via the first tube to the second balloon.
Example 27: The system of any example herein, in particular example 26, wherein the third balloon is configured to be in contact with the first balloon.
Example 28: The system of any example herein, in particular example 1, further comprising a sensor disposed within a right atrium or left atrium and configured to transmit signals to the first obstruction element in response to blood pressure changes.
Example 29: The system of any example herein, in particular example 28, further comprising a generator configured to supply power to the sensor.
Example 30: The system of any example herein, in particular example 29, wherein the generator is connected to the sensor via a wire.
Example 31: The system of any example herein, in particular example 28, further comprising a wire connecting the first obstruction element to the sensor.
Example 32: The system of any example herein, in particular example 28, wherein the first obstruction element is configured to form an orifice and to expand to at least partially close the orifice in response to signals from the sensor.
Example 33: The system of any example herein, in particular example 2, wherein the first balloon is configured to be disposed within the IVC and is configured to extend at least partially across an inflow junction of a hepatic vein.
Example 34: An implant comprising a first obstruction element disposed at least partially within an inferior vena cava (IVC), superior vena cava (SVC), or hepatic vein of a heart, the first obstruction element configured to expand and compress in response to pressure changes in the heart.
Example 35: The implant of any example herein, in particular example 34 wherein the first obstruction element comprises a first balloon.
Example 36: The implant of any example herein, in particular example 35, further comprising: a first tube; and a second balloon, wherein the first balloon is connected via the first tube to the second balloon disposed at least partially within a left atrium of the heart.
Example 37: The implant of any example herein, in particular example 36, wherein the first tube is configured to convey a liquid or gas between the first balloon and the second balloon.
Example 38: The implant of any example herein, in particular example 37, wherein the second balloon is configured to deflate in response to increased pressure in the left atrium and wherein the first balloon is configured to inflate in response to deflation of the second balloon.
Example 39: The implant of any example herein, in particular example 37, wherein the second balloon is configured to maintain an inflated state in response to pressure in the left atrium being below a threshold amount, and wherein the first balloon is configured to maintain a deflated state while the second balloon is in the inflated state.
Example 40: The implant of any example herein, in particular example 39, wherein the first balloon is disposed at least partially within the IVC.
Example 41: The implant of any example herein, in particular example 39, wherein the first balloon is disposed at least partially within the SVC.
Example 42: The implant of any example herein, in particular example 39, wherein the first tube is configured to extend at least partially through a coronary sinus of the heart between the first balloon and the second balloon.
Example 43: The implant of any example herein, in particular example 36, further comprising an anchor disposed at least partially within a right atrium, wherein the second balloon is attached to the anchor.
Example 44: The implant of any example herein, in particular example 43, wherein the anchor is a disc anchor.
Example 45: The implant of any example herein, in particular example 36, wherein the second balloon has a hemispherical shape and comprises a generally flat base configured to extend along a septal wall of the left atrium.
Example 46: The implant of any example herein, in particular example 35, further comprising a first stent disposed at least partially within the IVC.
Example 47: The implant of any example herein, in particular example 46, wherein the first stent is configured to at least partially enclose the first balloon.
Example 48: The implant of any example herein, in particular example 46, wherein the first stent comprises a wire frame and wherein the first stent is configured to allow blood flow through the wire frame.
Example 49: The implant of any example herein, in particular example 46, wherein the first stent has a cylindrical shape.
Example 50: The implant of any example herein, in particular example 46, wherein the first balloon is attached to an outer surface of a midsection of the first stent.
Example 51: The implant of any example herein, in particular example 50, wherein the first stent comprises a first end and a second end, and wherein a diameter of the midsection is less than diameters of the first end and second end.
Example 52: The implant of any example herein, in particular example 50, wherein the first balloon has a ring shape and forms a central orifice.
Example 53: The implant of any example herein, in particular example 52, further comprising a second stent at least partially enclosing the first balloon, wherein the first balloon is disposed between the first stent and the second stent.
Example 54: The implant of any example herein, in particular example 36, wherein the first balloon is disposed at least partially within a first hepatic vein, the implant further comprising a third balloon disposed in a second hepatic vein.
Example 55: The implant of any example herein, in particular example 54, further comprising a second tube, wherein the third balloon is connected via the second tube to the second balloon.
Example 56: The implant of any example herein, in particular example 55, wherein the first tube and the second tube branch from a main tube.
Example 57: The implant of any example herein, in particular example 36, further comprising: a second tube; and a third balloon disposed at least partially within the SVC, wherein the third balloon is connected via the second tube to the second balloon, and wherein the first balloon is disposed at least partially within the IVC or the hepatic vein.
Example 58: The implant of any example herein, in particular example 57, wherein the first tube and the second tube branch from a main tube.
Example 59: The implant of any example herein, in particular example 36, further comprising a third balloon disposed in the IVC or hepatic veins, wherein the third balloon is connected via the first tube to the second balloon.
Example 60: The implant of any example herein, in particular example 59, wherein the third balloon is configured to be in contact with the first balloon.
Example 61: The implant of any example herein, in particular example 34, further comprising a sensor disposed within a right atrium or left atrium and configured to transmit signals to the first obstruction element in response to blood pressure changes.
Example 62: The implant of any example herein, in particular example 61, further comprising a generator configured to supply power to the sensor.
Example 63: The implant of any example herein, in particular example 62, wherein the generator is connected to the sensor via a wire.
Example 64: The implant of any example herein, in particular example 61, further comprising a wire connecting the first obstruction element to the sensor.
Example 65: The implant of any example herein, in particular example 61, wherein the first obstruction element is configured to form an orifice and to expand to at least partially close the orifice in response to signals from the sensor.
Example 66: The implant of any example herein, in particular example 35, wherein the first balloon is configured to be disposed within the IVC and is configured to extend at least partially across an inflow junction of a hepatic vein.
Example 67: The implant of any example herein, in particular example 35, wherein the first balloon comprises a support device enclosed by a membrane of the first balloon.
Example 68: The implant of any example herein, in particular example 67, wherein the support device is configured to transition between an expanded configuration and a collapsed configuration in response to external pressure rising above or falling below one or more threshold pressures.
Example 69: The implant of any example herein, in particular example 67, wherein the support device comprises a platform at a midsection of the first balloon.
Example 70: The implant of any example herein, in particular example 69, wherein the platform is coupled to one or more legs comprising one or more joints to allow bending of the one or more legs.
Example 71: The implant of any example herein, in particular example 69, wherein the support device further comprises a flexible midsection between the platform and a base of the support device.
Example 72: The implant of any example herein, in particular example 68, wherein the support device comprises one or more features configured to resist transitioning between the expanded configuration and collapsed configuration.
Example 73: The implant of any example herein, in particular example 72, wherein the one or more features comprise one or more of cavities, pegs, mounds, notches, and dimples.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A system for modulating blood flow, the system comprising:
a first inflation device sized for placement at least partially within an inferior vena cava (IVC), superior vena cava (SVC), or hepatic vein of a heart, the first inflation device being flexible to allow the first inflation device to expand and compress;
a first tube attached to the first inflation device; and
a second inflation device connected to the first inflation device via the first tube, the second inflation device being sized for placement in a left atrium of the heart.

2. The system of claim 1, wherein the first inflation device comprises a first balloon and the second inflation device comprises a second balloon.

3. The system of claim 1, wherein the first tube comprises an inner lumen to convey a liquid or gas between the first inflation device and the second inflation device.

4. The system of claim 3, wherein the second inflation device is flexible to allow the second inflation device to compress in response to increased pressure in the left atrium and wherein the first inflation device is flexible to allow the first inflation device to inflate in response to deflation of the second inflation device.

5. The system of claim 1, wherein the first inflation device is sized for placement at least partially within a first hepatic vein, the system further comprising a third inflation device sized for placement in a second hepatic vein.

6. The system of claim 1, further comprising:
a second tube; and
a third inflation device sized for placement at least partially within the SVC,
wherein the third inflation device is connected via the second tube to the second inflation device, and wherein the first inflation device is sized for placement at least partially within the IVC or the hepatic vein.

7. The system of claim 1, further comprising a third inflation device sized for placement in the IVC or hepatic veins, wherein the third inflation device is connected via the first tube to the second inflation device.

8. The system of claim 1, further comprising a sensor sized for placement within a right atrium or left atrium and configured to transmit signals to the first inflation device in response to blood pressure changes.

9. An implant for modulating blood flow, the implant comprising:
a first inflation device disposed at least partially within an IVC, SVC, or hepatic vein of a heart, the first inflation device being flexible to allow the first inflation device to expand and compress;
a first tube attached to the first inflation device; and
a second inflation device connected to the first inflation device via the first tube, the second inflation device comprising a support device enclosed by a membrane of the second inflation device.

10. The implant of claim 9, wherein the first inflation device is sized for placement at least partially within a first hepatic vein, the implant further comprising a third inflation device sized for placement in a second hepatic vein.

11. The implant of claim 9, further comprising:
a second tube; and
a third inflation device sized for placement at least partially within the SVC,
wherein the third inflation device is connected via the second tube to the second inflation device, and wherein the first inflation device is sized for placement at least partially within the IVC or the hepatic vein.

12. The implant of claim 9, wherein the support device includes joints to allow the support device to transition between an expanded configuration and a collapsed configuration in response to external pressure.

13. The implant of claim 9, wherein the second inflation device is sized for placement within a left atrium.

14. An implant for modulating blood flow into a blood vessel from a branching blood vessel, the implant comprising:
a stent sized for placement within a blood vessel at or near a junction of a branching blood vessel, the stent comprising an inner lumen to allow blood flow through the stent and being flexible to expand and compress in width; and
a generator connected to the stent via one or more wires to cause expansion or compression of the stent in response to sensed blood pressure.

15. The implant of claim 14, wherein the stent comprises a first inflation device extending along an outer or inner surface of a frame of the stent and being at least partially hollow to allow the first inflation device to fill with gas or fluid to expand and obstruct flow from the branching blood vessel and compress to allow flow from the branching blood vessel.

16. The implant of claim 14, further comprising a sensor connected to the stent via one or more wires and sized for placement within a chamber of a heart to sense blood pressure within the chamber of the heart.

17. The implant of claim 16, wherein the generator provides electrical power to the sensor to allow the sensor to sense pressure changes and transmit electric current to the stent.

18. The implant of claim 14, wherein the stent comprises a midsection between a first end portion and a second end portion of the stent, and wherein the midsection has a reduced diameter relative to the first end portion and the second end portion.

19. The implant of claim 18, wherein the stent comprises a first inflation device extending along the midsection and not along the first end portion and the second end portion.

20. The implant of claim 14, wherein the generator is subcutaneous.
